SEQUENCE LISTING (1 sequence)

SEQ ID NO: 1 - protein, 229 aa, Homo sapiens
MQDEDGYITLNIKTRKPALISVGSASSSWWRVMALILLILCVGMVVGLVALGIWSVMQRNYLQGENENRTGTLQQLAKRFCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTFCENKHYLMCERKAGMTKVDQLP

(19) United States
(12) Patent Application Publication
(10) Pub. No.: US 2014/0227265 A1
(43) Pub. Date: Aug. 14, 2014

(54) METHOD OF TREATING OR AMELIORATING METABOLIC DISORDERS USING CLEC-2

(71) Applicants: Xinle Wu, San Diego, CA (US); Yang Li, Mountain View, CA (US)

(72) Inventors: Xinle Wu, San Diego, CA (US); Yang Li, Mountain View, CA (US)

(73) Assignee: AMGEN INC., Thousand Oaks, CA (US)

(21) Appl. No.: 14/127,105

(22) PCT Filed: Jun. 18, 2012
(86) PCT No.: PCT/US2012/042960

§ 371 Date: Apr. 9, 2014

Related U.S. Application Data
(60) Provisional application No. 61/498,370, filed on Jun. 17, 2011.

(51) Int. Cl. C07K 14/705; C07K 16/28

(52) U.S. Cl. CPC C07K 14/7056; C07K 16/2851; A61K 2039/505
USPC 424/134.1; 514/6.9; 514/6.7

(57) ABSTRACT

Methods of treating metabolic diseases and disorders using a Clec-2 extracellular domain are provided. In various embodiments the metabolic disease or disorder is type 2 diabetes, elevated glucose levels, elevated insulin levels and elevated triglyceride levels.

This application claims the benefit of U.S. Provisional Application No. 61/498,370 filed Jun. 17, 2011, which is incorporated in its entirety by reference herein.

### FIELD OF THE INVENTION

The disclosed invention relates to the treatment or amelioration of a metabolic disorder, such as diabetes, elevated glucose levels, elevated insulin levels or elevated triglyceride, and insulin resistance by administering a therapeutically effective amount of a molecule comprising the Clec-2 extracellular domain or a fragment thereof or an antibody against Clec-2 to a subject in need thereof.

### BACKGROUND OF THE INVENTION

CLEC-2 was identified using a bio-informatic approach to identify molecules with similarity to C-type lectin-like receptors that were known to be involved in protein—protein recognition interactions in the immune system (M. Colonna et al., 2000, Eur J Immunol 30 pp. 697-704). CLEC-2 was identified at the transcript level in peripheral blood cells, bone marrow, myeloid cells (monocytes, dendritic cells and granulocytes), natural killer cells and liver. The gene encoding CLEC-2 is located within a cluster of related genes, including DECTIN-1 and LOX-1 on human chromosome 12 (Sobanov, A. et al. 2001, Eur J Immunol, 31, pp. 3493-3503.) CLEC-2 is also referred to as C-type lection domain family 1 member B or CLEC1B.

CLEC-2 is a member of the C-type lectin-like family of proteins. It has recently been identified as a receptor on the surface of platelets. (K. Suzuki-Inoue et al., 2006, Blood, 107, pp. 542-549.) Ligand binding by CLEC-2 promotes phosphorylation of a tyrosine in the cytoplasmic domain YXXL motif of CLEC-2 by Src kinases and further downstream signaling events trigger platelet activation and aggregation (See K. Suzuki-Inoue et al, supra). The Malayan pit viper Calloselasma rhodostoma produces a potent venom protein, rhodocytin, which elicits powerful platelet activation and aggregation. Rhodocytin was recently shown to be a ligand for CLEC-2, a newly identified receptor on the surface of platelets, and binding of rhodocytin to CLEC-2 triggers a novel platelet-signaling pathway. Rhodocytin binding leads to tyrosine phosphorylation in the cytoplasmic tail of CLEC-2, which promotes the binding of spleen tyrosine kinase (Syk), subsequent activation of PLCγ2, and platelet activation and aggregation.

### SUMMARY OF THE INVENTION

A method of treating a metabolic condition is provided. In one embodiment a method of treating a metabolic disorder in a subject, comprising administering to the subject a therapeutically effective amount of a Clec-2 inhibitor is provided. In one embodiment the metabolic condition is diabetes, particularly type II diabetes, an elevated glucose level, an elevated insulin level, an elevated triglyceride level, insulin resistance or poor oral glucose tolerance.

In one embodiment of the invention the Clec-2 inhibitor comprises the Clec-2 receptor, particularly the extracellular domain of the Clec-2 receptor or a fragment thereof. In one embodiment the extracellular domain of the Clec-2 receptor or a fragment thereof is a human CLEC2 extracellular domain or a fragment thereof. The extracellular domain of the Clec-2 receptor or fragment thereof may be modified to increase half life in a subject. In one embodiment the half life is increase by conjugating an immunoglobulin constant region or fragment thereof to the CLEC2 extracellular domain. In one embodiment the immunoglobulin constant region is a human immunoglobulin constant region or fragment thereof. In some embodiments, other half life extending modalities may be used to modify the Clec-2 extracellular domain or fragments thereof such as, conjugation to human serum albumin, conjugation to human serum albumin binders, pegylation, pegylation mimetics, etc.

In some embodiments, the Clec-2 inhibitor of the invention comprises a polypeptide encoded by a polynucleotide which comprises a sequence according to SEQ ID 2, 4, 6 or 13 or that comprises at least 90%, 95% or 98% sequence identity with that of SEQ ID 2, 4, 6 or 13. In some embodiments the Clec-2 inhibitor of the invention comprises a polypeptide having the amino acid sequence of SEQ ID 1, 3, 5 or 12 or that comprises at least 90%, 95% or 98% with that of SEQ ID 1, 3, 5 or 12.

In some embodiments, Clec-2 inhibitor of the invention is an antibody or fragment thereof that specifically binds to Clec-2 or a ligand of Clec-2. In some embodiments of the invention, the antibody or fragment thereof specifically binds to human Clec-2 receptor. In some embodiments, the antibody or fragment thereof specifically binds to a polypeptide comprising the amino acid sequence according to SEQ ID 1, 3, 5, 7 or 9. In some embodiments, the antibody of the invention or fragment thereof is a monoclonal antibody or fragment thereof. In some embodiments, the antibody of the invention is a human, humanized or chimeric antibody.

In some embodiments of the invention, the subject to be treated is a mammal, particularly a human. In some embodiments of the invention the CLEC2 inhibitor is administered in the form of a pharmaceutical composition comprising a therapeutically effective amount of the Clec-2 inhibitor in admixture with a pharmaceutically-acceptable carrier.

In some embodiments, the subject to be treated by the methods of the invention comprises a glucose level at a time point subsequent to administration of the Clec-2 inhibitor to the subject that is lower than at a time point prior to administration of the Clec-2 inhibitor. In some embodiments the subject to be treated will comprise a blood glucose level, particularly a fasting blood glucose level, of lower than 400, 300, 200, 150 or 140 mg/dl but not lower than about 60 mg/dl following treatment with a therapeutically effective amount of the Clec-2 inhibitor of the invention.

In some embodiments, the subject to be treated by the methods of the invention comprises an insulin level at a time point subsequent to administration of the Clec-2 inhibitor to the subject that is lower than at the time point prior to the administration. In some embodiments the subject's insulin level at the time point subsequent to Clec-2 inhibitor administration is at least 5%, 10% or 15% lower than the subject's insulin level prior to administration.

In some embodiments, the subject's insulin resistance is improved at a time point subsequent to administration of the Clec-2 inhibitor to the subject that is improved compared to a time point prior to the administration. In some embodiments, subject's glucose level is the subject's blood glucose level, particularly fasting blood glucose level. In some embodiments, the subject's insulin level is the subject's plasma insulin level.

In some embodiments, the subject to be treated by the methods of the invention comprises a triglyceride level at a time point subsequent to administration of the Clec-2 inhibitor to the subject that is lower than at a time point prior to the administration. In some embodiments, the subject's triglyeride level is the subject's blood triglyceride level. In some embodiments the subject's triglyceride level at the time point subsequent to Clec-2 inhibitor administration is at least 5%, 10% or 15% lower than the subject's triglyceride level prior to administration

In some embodiments, the subject treated by the methods of the invention comprises an oral glucose tolerance that is improved at a time point subsequent to administration of the Clec-2 inhibitor than at a time point prior to the administration.

In one embodiment of the invention a method of treating a metabolic condition in a subject, comprising administering to the subject a therapeutically effective amount of the extracellular domain of the Clec-2 receptor or a fragment thereof. In one embodiment, the metabolic condition to be treated is type 2 diabetes, an elevated glucose level, an elevated insulin level, an elevated triglyceride level, insulin resistance or poor oral glucose tolerance.

In one embodiment, the extracellular domain of the Clec-2 receptor or fragment thereof is modified to increase half life in a subject. In one embodiment, extracellular domain of the Clec-2 receptor or fragment thereof is conjugated to an immuglobulin constant region or fragment thereof. In one embodiment, the extracellular domain of the Clec-2 receptor or fragment thereof is a human extracellular domain or fragment thereof. In one embodiment, the immunoglobulin constant region is a human immunoglobulin constant region or fragment thereof.

In some embodiments, the Clec-2 extracellular domain comprises a polypeptide encoded by a polynucleotide which comprises a sequence of SEQ ID 2, 4, 6 or 13 or a sequence having at least 90%, 95% or 98% sequence identity with that of SEQ ID 2, 4, 6 or 13. In some embodiments, the Clec-2 extracellular domain comprises a polypeptide comprising an amino acid sequence of SEQ ID 1, 3, 5 or 12 or an amino acid sequence that is at least 90%, 95% or 98% identical to the amino acid sequence of SEQ ID 1, 3, 5 or 12.

In some embodiments of method of the invention, the subject is a mammal, particularly a human. In some embodiments of the invention, the Clec-2 extracellular domain or fragment thereof is administered in the form of a pharmaceutical composition comprising the Clec-2 extracellular domain in admixture with a pharmaceutically-acceptable carrier. In some embodiments, the subject to be treated by the methods of the invention comprises: a) a glucose level at a time point subsequent to administration of the Clec-2 extracellular domain to the subject that is lower than at a time point prior to administration of the Clec-2 extracellular domain; b) an insulin level at a time point subsequent to administration of the Clec-2 extracellular domain or fragment thereof to the subject that is lower than at the time point prior to the administration; or c) a triglyceride level at a time point subsequent to administration of the Clec-2 extracellular domain or fragment thereof to the subject that is lower than at the time point prior to the administration.

In some embodiments, insulin resistance in a subject treated by the methods of the invention is improved at a time point subsequent to administration of the Clec-2 extracellular domain or fragment thereof to the subject that is improved compared to a time point prior to the administration. In some embodiments, the subject's glucose level is the subject's blood glucose level. In some embodiments, the subject's insulin level is the subject's plasma insulin level. In some embodiments, the subject's triglyceride level is the subject's blood triglyceride level.

In some embodiments, the subject to be treated by the methods of the invention comprises an oral glucose tolerance that is improved at a time point subsequent to administration of the Clec-2 extracellular domain or fragment thereof than at a time point prior to the administration.

### BRIEF DESCRIPTION OF THE DRAWINGS

FIG. 1 is a bar graph showing the serum protein level of hFc-mCLEC2(ECD) in DIO mice on day 9 and day 23 after injection.

FIG. 2 is a bar graph showing the effect of hFc-mCLEC2(ECD) on body weight in DIO mice compared to that of the control animals treated with empty AAV.

FIG. 3 is a bar graph showing lower baseline glucose levels in DIO mice treated with hFc-mCLEC2(ECD) compared to those of the control animals.

FIG. 4 is a bar graph showing a lower insulin level in DIO mice injected with hFc-mCLEC2(ECD) 40 days post AAV injection as compared that of the control mice.

FIG. 5 is a plot showing improved oral glucose tolerance in hFc-mCLEC2 (ECD) treated mice on day 23 post AAV injection as compared to that of the control DIO mice.

FIG. 6 is a bar graph showing the serum protein level of hFc-mCLEC2(ECD) in DIO mice on day 7, 13 and 18 after HTV injection of the DNA construct.

FIG. 7 is a bar graph showing the effect of hFc-mCLEC2(ECD) on body weight in DIO mice compared to that of control animals.

FIG. 8 is a bar graph showing the baseline glucose level two days prior to HTV injections and on days 7 and 13 following injection in DIO mice treated with hFc-mCLEC2(ECD) as compared to those in the control animals.

FIG. 9 is a bar graph showing a lower insulin level in DIO mice injected with hFc-mCLEC2(ECD) on day 40 following the HTV injection as compared to that control DIO mice.

FIG. 10 is a plot showing improved oral glucose tolerance in hFc-mCLEC2 (ECD) treated mice on day 13 post HTV injection as compared to that of control DIO mice.

FIG. 11 is a bar graph showing a lower liver triglyceride level in hFc-mCLEC2 (ECD) treated mice on day 18 post HTV injection as compared to that in control DIO mice.

FIG. 12 is a bar graph showing lower insulin levels on day 8 post injection in DIO mice treated with 10 mg/kg or 30 mg/kg of recombinant hFc-mCLEC2 (ECD) as compared to the insulin level in the control animals.

FIG. 13 is a plot showing improved oral glucose tolerance DIO mice treated with 10 mg/kg or 30 mg/kg recombinant hFc-mCLEC2 (ECD) on day 13 post as compared to that of control DIO mice.

FIG. 14 is a bar graph showing the baseline glucose level two days prior to AAV injections and on days 12 and 26 following injection in ob/ob mice treated with hFc-mCLEC2(ECD) as compared to those in the control animals.

FIG. 15 is a plot showing improved oral glucose tolerance in hFc-mCLEC2 (ECD) treated mice on day 12 post AAV injection as compared to that of control ob/ob mice.

FIG. 16 is a is a bar graph showing the serum protein level of hFc-mCLEC2(ECD) in ob/ob mice on day 7 and day 14 after injection of the recombinant protein.

FIG. 17 is a plot showing improved oral glucose tolerance in hFc-mCLEC2 (ECD) treated mice on day 12 post injection of the recombinant protein as compared to that of control ob/ob mice.

### DETAILED DESCRIPTION OF THE INVENTION

The instant disclosure provides a method of treating a metabolic disorder, such as diabetes, including type 2 diabetes, elevated glucose levels, elevated insulin levels, or elevated triglyceride levels by administering to a subject in need thereof a therapeutically effective amount of a molecule comprising a CLEC2 polypeptide. Methods of administration and delivery are also provided.

Recombinant polypeptide and nucleic acid methods used herein, including in the Examples, are generally those set forth in Sambrook et al., Molecular Cloning: A Laboratory Manual (Cold Spring Harbor Laboratory Press, 1989) or Current Protocols in Molecular Biology (Ausubel et al., eds., Green Publishers Inc. and Wiley and Sons 1994), both of which are incorporated herein by reference for any purpose.

### I. GENERAL DEFINITIONS

Following convention, as used herein “a” and “an” mean “one or more” unless specifically indicated otherwise.

The term “antibody” refers to an intact immunoglobulin of any isotype, or a fragment thereof that can compete with the intact antibody for specific binding to the target antigen, and includes chimeric, humanized, fully human, and bispecific antibodies. An intact antibody generally will comprise at least two full-length heavy chains and two full-length light chains, but in some instances may include fewer chains such as antibodies naturally occurring in camelids which may comprise only heavy chains. Antibodies according to the invention may be derived solely from a single source, or may be “chimeric,” that is, different portions of the antibody may be derived from two different antibodies. For example, the CDR regions may be derived from a rat or murine source, while the framework region of the V region are derived from a different animal source, such as a human. The antibodies or binding fragments of the invention may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains according to the invention include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the —COOH end. Heavy chains according to the invention may be of any isotype, including IgG (including IgG1, IgG2, IgG3 and IgG4 subtypes), IgA (including IgA1 and IgA2 subtypes), IgM and IgE.

The term “immunologically functional fragment” (or simply “fragment”) of an immunoglobulin chain, as used herein, refers to a portion of an antibody light chain or heavy chain that lacks at least some of the amino acids present in a full-length chain but which is capable of binding specifically to an antigen. Such fragments are biologically active in that they bind specifically to the target antigen and can compete with intact antibodies for specific binding to a given epitope. In one aspect of the invention, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of intact antibodies. Immunologically functional immunoglobulin fragments of the invention include, but are not limited to, Fab, Fab′, F(ab′)2, Fv, domain antibodies and single-chain antibodies, and may be derived from any mammalian source, including but not limited to human, mouse, rat, camelid or rabbit. It is contemplated further that a functional portion of the inventive antibodies, for example, one or more CDRs, could be covalently bound to a second protein or to a small molecule to create a therapeutic agent directed to a particular target in the body, possessing bifunctional therapeutic properties, or having a prolonged serum half-life.

The term “neutralizing antibody” refers to an antibody that binds to a ligand, prevents binding of the ligand to its binding partner and interrupts the biological response that otherwise would result from the ligand binding to its binding partner. In assessing the binding and specificity of an antibody or immunologically functional fragment thereof, an antibody or fragment will substantially inhibit binding of a ligand to its binding partner when an excess of antibody reduces the quantity of binding partner bound to the ligand by at least about 20%, 30%, 40%, 50%, 60%, 70%, 80%, 85%, 90%, 95%, 97%, 99% or more (as measured in an in vitro competitive binding assay). In the case of CLEC2 antibodies, a neutralizing antibody will inhibit signaling through the CLEC2 pathway by either binding the CLEC2 receptor and preventing ligand binding to the receptor or by binding the ligand and prevent it from binding to the CLEC2 receptor.

An antibody of the invention is said to “specifically bind” its target antigen when the dissociation constant (K_{d}) is 1×10⁻⁸ M. The antibody specifically binds antigen with “high affinity” when the Kd is 5×10⁻⁹ M, and with “very high affinity” when the Kd is 5×10⁻¹⁰ M. In one embodiment of the invention, the antibody has a Kd of 1×10⁻⁹ M and an off-rate of about 1×10⁻⁴/sec. In one embodiment of the invention, the off-rate is <1×10⁻⁵. In other embodiments of the invention, the antibodies will bind to human DKK1 with a Kd of between about 1×10⁻⁸ M and 1×10⁻¹⁰ M, and in yet another embodiment it will bind with a Kd 2×10⁻¹⁰. One of skill in the art will recognize that specifically binding does not mean exclusive binding, rather it allows for some degree of non-specific binding as is typical in biological reactions between groups with affinity to one another.

As used herein, the terms “amino acid” and “residue” are interchangeable and, when used in the context of a peptide or polypeptide, refer to both naturally occurring and synthetic amino acids, as well as amino acid analogs, amino acid mimetics and non-naturally occurring amino acids that are chemically similar to the naturally occurring amino acids.

A “naturally occurring amino acid” is an amino acid that is encoded by the genetic code, as well as those amino acids that are encoded by the genetic code that are modified after synthesis, e.g., hydroxyproline, γ-carboxyglutamate, and O-phosphoserine. An amino acid analog is a compound that has the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but will retain the same basic chemical structure as a naturally occurring amino acid.

An “amino acid mimetic” is a chemical compound that has a structure that is different from the general chemical structure of an amino acid, but that functions in a manner similar to a naturally occurring amino acid. Examples include a methacryloyl or acryloyl derivative of an amide, β-, γ-, δ-imino acids (such as piperidine-4-carboxylic acid) and the like.

A “non-naturally occurring amino acid” is a compound that has the same basic chemical structure as a naturally occurring amino acid, but is not incorporated into a growing polypeptide chain by the translation complex. “Non-naturally occurring amino acid” also includes, but is not limited to, amino acids that occur by modification (e.g., posttranslational modifications) of a naturally encoded amino acid (including but not limited to, the 20 common amino acids) but are not themselves naturally incorporated into a growing polypeptide chain by the translation complex. A non-limiting lists of examples of non-naturally occurring amino acids that can be inserted into a polypeptide sequence or substituted for a wild-type residue in polypeptide sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVa1), N-α-methyl leucine (NMeLeu), Nα-methylnorleucine (NMeN1e), cyclopentylglycine (Cpg), cyclohexylglycine (Chg), acetylarginine (acetylarg), α, β-diaminopropionoic acid (Dpr), α, γ-diaminobutyric acid (Dab), diaminopropionic acid (Dap), cyclohexylalanine (Cha), 4-methyl-phenylalanine (MePhe), β, β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

The term “isolated nucleic acid molecule” refers to a single or double-stranded polymer of deoxyribonucleotide or ribonucleotide bases read from the 5′ to the 3′ end (e.g., Clec-2 extracellular domain nucleic acid sequence provided herein), or an analog thereof, that has been separated from at least about 50 percent of polypeptides, peptides, lipids, carbohydrates, polynucleotides or other materials with which the nucleic acid is naturally found when total nucleic acid is isolated from the source cells. Preferably, an isolated nucleic acid molecule is substantially free from any other contaminating nucleic acid molecules or other molecules that are found in the natural environment of the nucleic acid that would interfere with its use in polypeptide production or its therapeutic, diagnostic, prophylactic or research use.

The term “isolated polypeptide” refers to a polypeptide (e.g., a CLEC2 extracellular domain polypeptide sequence provided herein) that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic or research use.

The term “encoding” refers to a polynucleotide sequence encoding one or more amino acids. The term does not require a start or stop codon. An amino acid sequence can be encoded in any one of six different reading frames provided by a polynucleotide sequence.

The terms “identical” and percent “identity,” in the context of two or more nucleic acids or polypeptide sequences, refer to two or more sequences or subsequences that are the same. “Percent identity” means the percent of identical residues between the amino acids or nucleotides in the compared molecules and is calculated based on the size of the smallest of the molecules being compared. For these calculations, gaps in alignments (if any) can be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), (1988) New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., (1987) Sequence Analysis in Molecular Biology, New York: Academic Press; Sequence Analysis Primer, (Gribskov, M. and Devereux, J., eds.), 1991, New York: M. Stockton Press; and Carillo et al., (1988) SIAM J. Applied Math. 48:1073.

In calculating percent identity, the sequences being compared are aligned in a way that gives the largest match between the sequences. The computer program used to determine percent identity is the GCG program package, which includes GAP (Devereux et al., (1984) Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., (1978) Atlas of Protein Sequence and Structure 5:345-352 for the PAM 250 comparison matrix; Henikoff et al., (1992) Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program are the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences can result in matching of only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans at least 50 contiguous amino acids of the target polypeptide.

The terms “Clec-2 inhibitor” or “CLEC2 inhibitor” refer to a molecule having an inhibitory effect on signaling through the Clec-2 pathway. A Clec-2 inhibitor may comprise a neutralizing antibody that binds to the Clec-2 receptor or that binds to a ligand of the Clec-2 receptor or that binds to a receptor for the Clec-2 extracellular domain. A Clec-2 inhibitor may also comprise a molecule that includes a portion of the Clec-2 receptor, particularly the extracellular domain or fragment thereof of the human Clec-2 receptor.

The terms “CLEC2 polypeptide” and “CLEC2 protein” and “Clec-2 polypeptide” and “Clec-2 protein” and“CLEC2 receptor” and “Clec-2 receptor” are used interchangeably and mean a naturally-occurring wild-type polypeptide expressed in a mammal, such as a human or a mouse. For purposes of this disclosure, the terms “CLEC2 polypeptide” and “CLEC2 protein” and “Clec-2 polypeptide” and “Clec-2 protein” and“CLEC2 receptor” and “Clec-2 receptor can be used interchangeably to refer to full length human isotype 1 Clec-2 polypeptide, e.g. SEQ ID 1 which consists of 229 amino acid residues and which is encoded by the nucleic acid sequence of SEQ ID 2, any form comprising the extracellular domain e.g. SEQ ID 3 which consists of 179 amino acids and is encoded by the nucleic acid sequence of SEQ ID 4 and in which the cytoplasmic domain (residues 1-33 of SEQ ID) and the transmembrane domain (residues 34-54 of SEQ ID 1) have been removed, any form of full length human isotype 2, e.g. SEQ ID 5 which consists of 196 residues and is encoded by the nucleic acid sequence of SEQ ID 6, and any form of the CLEC2 polypeptide comprising the extracellular domain from which the intracellular and transmembrane domains have been removed. CLEC2 polypeptides can but need not comprise an amino terminal methionine which may be introduced by engineering or as a result of a bacterial expression process.

The term “CLEC2 polypeptide” also encompasses a CLEC2 polypeptide in which a naturally occurring CLEC2 polypeptide sequence (e.g., SEQ ID NOs 1, 3 or 5) has been modified. Such modifications include, but are not limited to, one or more amino acid substitutions, including substitutions with non-naturally occurring amino acids non-naturally-occurring amino acid analogs and amino acid mimetics.

In various embodiments, a CLEC2 polypeptide comprises an amino acid sequence that is at least about 85 percent identical to a naturally-occurring CLEC2 polypeptide (e.g., SEQ ID NOs:1, 3 or 5). In other embodiments, a CLEC2 polypeptide comprises an amino acid sequence that is at least about 90 percent, or about 95, 96, 97, 98, or 99 percent identical to a naturally-occurring CLEC2 polypeptide amino acid sequence (e.g., SEQ ID NOs:1, 3 or 5). Such CLEC2 polypeptides preferably, but need not, possess at least one activity of a wild-type CLEC2 polypeptide, such as the ability to lower blood glucose, insulin, or triglyceride levels, or the ability to improve glucose tolerance. The present invention also encompasses nucleic acid molecules encoding such CLEC2 polypeptide sequences.

As stated herein, a CLEC2 polypeptide can comprise the intracellular and transmembrane domains (residues 1-54 of SEQ ID 3) or it can have the intracellular and transmembrane domain sequence removed (providing SEQ ID 3). The naturally-occurring biologically active form of the CLEC2 polypeptide is a homodimer. In some instances, a CLEC2 polypeptide can be used to treat or ameliorate a metabolic disorder in a subject and comprises the extracelluar domain of the mature form of CLEC2 polypeptide that is derived from the same species as the subject. In some embodiments the CLEC2 polypeptide comprises an alteration to extend serum half-life of the CLEC2 polypeptide. For example, in some embodiments the extracellular domain is a fused to the constant region of an immunoglobulin using methods known in the art.

In one embodiment the CLEC2 polypeptide comprises the Clec-2 human extracellular domain fused to a human immunoglobulin constant region and comprises the 415 amino acid sequence below:

| SEQ ID No. 12 | |
|---|---|
| 1 | MEWSWVFLFF LSVTTGVHSD KTHTCPPCPA PELLGGPSVF LFPPKPKDTL MISRTPEVTC |
| 61 | VVVDVSHEDP EVKFNWYVDG VEVHNAKTKP REEQYNSTYR VVSVLTVLHQ DWLNGKEYKC |
| 121 | KVSNKALPAP IEKTISKAKG QPREPQVYTL PPSRDELTKN QVSLTCLVKG FYPSDIAVEW |
| 181 | ESNGQPENNY KTTPPVLDSD GSFFLYSKLT VDKSRWQQGN VFSCSVMHEA LHNHYTQKSL |
| 241 | SLSPGKGGGG SGGGGASGIW SVMQRNCQYV VKQSELKGTF KGHKCSPCDT NWRYYGDSCY |
| 301 | GFFRHNLTWE ESKQYCTDMN ATLLKIDNRN IVEYIKARTH LIRWVGLSRQ KSNEVWKWED |
| 361 | GSVISENMFE FLEDGKGNMN CAYFHNGKMH PTFCENKHYL MCERKAGMTK VDQLP |

that is encoded by the DNA sequence:

SEQ ID 13
ATGGAATGGA GCTGGGTCTT TCTCTTCTTC CTGTCAGTAA
CTACAGGTGT CCACTCCGAC AAAACTCACA CATGCCCACC
GTGCCCAGCA CCTGAACTCC TGGGGGGACC GTCAGTCTTC
CTCTTCCCCC CAAAACCCAA GGACACCCTC ATGATCTCCC
GGACCCCTGA GGTCACATGC GTGGTGGTGG ACGTGAGCCA
CGAAGACCCT GAGGTCAAGT TCAACTGGTA CGTGGACGGC
GTGGAGGTGC ATAATGCCAA GACAAAGCCG CGGGAGGAGC
AGTACAACAG CACGTACCGT GTGGTCAGCG TCCTCACCGT
CCTGCACCAG GACTGGCTGA ATGGCAAGGA GTACAAGTGC
AAGGTCTCCA ACAAAGCCCT CCCAGCCCCC ATCGAGAAAA
CCATCTCCAA AGCCAAAGGG CAGCCCCGAG AGCCACAGGT
GTACACCCTG CCCCCATCCC GGGATGAGCT GACCAAGAAC
CAGGTCAGCC TGACCTGCCT GGTCAAAGGC TTCTATCCCA
GCGACATCGC CGTGGAGTGG GAGAGCAATG GGCAGCCGGA
GAACAACTAC AAGACCACGC CTCCCGTGCT GGACTCCGAC
GGCTCCTTCT TCCTCTACAG CAAGCTCACC GTGGACAAGA
GCAGGTGGCA GCAGGGGAAC GTCTTCTCAT GCTCCGTGAT
GCATGAGGCT CTGCACAACC ACTACACGCA GAAGAGCCTC
TCCCTGTCTC CGGGTAAAGG AGGCGGTGGA TCTGGCGGAG
GTGGAGCTAG CGGGATTTGG TCTGTCATGC AGCGCAATTA
CCTACAAGGT GAGAATGAAA ATCGCACAGG AACTCTGCAA
CAATTAGCAA AGCGCTTCTG TCAATATGTG GTAAAACAAT
CAGAACTAAA GGGCACTTTC AAAGGTCATA AATGCAGCCC
CTGTGACACA AACTGGAGAT ATTATGGAGA TAGCTGCTAT
GGGTTCTTCA GGCACAACTT AACATGGGAA GAGAGTAAGC
AGTACTGCAC TGACATGAAT GCTACTCTCC TGAAGATTGA
CAACCGGAAC ATTGTGGAGT ACATCAAAGC CAGGACTCAT
TTAATTCGTT GGGTCGGATT ATCTCGCCAG AAGTCGAATG
AGGTCTGGAA GTGGGAGGAT GGCTCGGTTA TCTCAGAAAA
TATGTTTGAG TTTTTGGAAG ATGGAAAAGG AAATATGAAT
TGTGCTTATT TTCATAATGG GAAAATGCAC CCTACCTTCT
GTGAGAACAA ACATTATTTA ATGTGTGAGA GGAAGGCTGG
CATGACCAAG GTGGACCAAC TACCTTAA.

A polypeptide is preferably biologically active. In various respective embodiments, a CLEC2 polypeptide has a biological activity that is equivalent to, greater to or less than that of the naturally occurring form of the extracellular domain of the mature CLEC2 protein from which the cytoplasmic and transmembrane domains have been removed from the full length CLEC2 sequence. Examples of biological activities include the ability to lower blood glucose, insulin, or triglyceride levels; or the ability to improve glucose tolerance.

The terms “therapeutically effective dose” and “therapeutically effective amount,” as used herein, means an amount of a CLEC2 polypeptide or CLEC2 antibody that specifically binds the CLEC2 polypeptide that elicits a biological or medicinal response in a tissue system, animal, or human being sought by a researcher, physician, or other clinician, which includes alleviation or amelioration of the symptoms of the disease or disorder being treated, i.e., an amount of a CLEC2 polypeptide or antibody that specifically binds the CLEC2 polypeptide that supports an observable level of one or more desired biological or medicinal response, for example lowering blood glucose, insulin or triglyceride levels; or improving glucose tolerance or insulin sensitivity.

The term “elevated” as used herein means a level that is greater than the threshold established as normal by the medical community. For example, in some embodiments a subject to be treated will comprise an elevated glucose level, particularly an elevated blood glucose level of 100 mg/dl or greater, particularly of 126 mg/dl or greater. In some embodiments a subject to be treated will comprise an elevated insulin level, particularly an elevated plasma insulin level of 20 mU/l or greater, particularly of 24. 9 mU/l or greater. In some embodiments a subject to be treated will comprise an elevated triglyceride level, particularly an elevated blood triglyceride level of 175 mg/dl or greater, particularly of 200 mg/dl or greater.

The term “improved as used herein means that a response or condition in a subject is better after administration of the CLEC2 polypeptide or CLEC2 antibody than the response in the subject before administration. For example, in an OGTT test an improved response would be that the blood glucose level would be lower at a time point after administration of the CLEC2 polypeptide or CLEC2 antibody as compared to blood glucose levels in the OGTT test prior to the first administration of the CLEC2 polypeptide or CLEC2 antibody.

### II. CLEC2 POLYPEPTIDES AND NUCLEIC ACIDS

As disclosed herein, a CLEC2 polypeptide described by the instant disclosure can be engineered and/or produced using standard molecular biology methodology. In various examples, a nucleic acid sequence encoding a CLEC2, which can comprise all or a portion of SEQ IDs 1, 3 or 5 can be isolated and/or amplified from genomic DNA, or cDNA using appropriate oligonucleotide primers. Primers can be designed based on the nucleic and amino acid sequences provided herein according to standard (RT)-PCR amplification techniques. The amplified CLEC2 nucleic acid can then be cloned into a suitable vector and characterized by DNA sequence analysis.

Oligonucleotides for use as probes in isolating or amplifying all or a portion of the CLEC2 sequences provided herein can be designed and generated using standard synthetic techniques, e.g., automated DNA synthesis apparatus, or can be isolated from a longer sequence of DNA.

### II.A. Naturally-Occurring and Variant Clec2 Polypeptide and Polynucleotide Sequences

In vivo, CLEC2 occurs in two isoform. The 229 amino acid sequence of full length human CLEC2 isoform 1 is:

(SEQ ID 1)
MQDEDGYITL NIKTRKPALI SVGSASSSWW RVMALILLIL
CVGMVVGLVA LGIWSVMQRN YLQGENENRT GTLQQLAKRF
CQYVVKQSEL KGTFKGHKCS PCDTNWRYYG DSCYGFFRHN
LTWEESKQYC TDMNATLLKI DNRNIVEYIK ARTHLIRWVG
LSRQKSNEVW KWEDGSVISE NMFEFLEDGK GNMNCAYFHN
GKMHPTFCEN KHYLMCERKA GMTKVDQLP

and is encoded by the DNA sequence:

(SEQ ID 2)
ATGCAGGATG AAGATGGATA CATCACCTTA AATATTAAAA
CTCGGAAACC AGCTCTCATC TCCGTTGGCT CTGCATCCTC
CTCCTGGTGG CGTGTGATGG CTTTGATTCT GCTGATCCTG
TGCGTGGGGA TGGTTGTCGG GCTGGTGGCT CTGGGGATTT
GGTCTGTCAT GCAGCGCAAT TACCTACAAG GTGAGAATGA
AAATCGCACA GGAACTCTGC AACAATTAGC AAAGCGCTTC
TGTCAATATG TGGTAAAACA ATCAGAACTA AAGGGCACTT
TCAAAGGTCA TAAATGCAGC CCCTGTGACA CAAACTGGAG
ATATTATGGA GATAGCTGCT ATGGGTTCTT CAGGCACAAC
TTAACATGGG AAGAGAGTAA GCAGTACTGC ACTGACATGA
ATGCTACTCT CCTGAAGATT GACAACCGGA ACATTGTGGA
GTACATCAAA GCCAGGACTC ATTTAATTCG TTGGGTCGGA
TTATCTCGCC AGAAGTCGAA TGAGGTCTGG AAGTGGGAGG
ATGGCTCGGT TATCTCAGAA AATATGTTTG AGTTTTTGGA
AGATGGAAAA GGAAATATGA ATTGTGCTTA TTTTCATAAT
GGGAAAATGC ACCCTACCTT CTGTGAGAAC AAACATTATT
TAATGTGTGA GAGGAAGGCT GGCATGACCA AGGTGGACCA
ACTACCTTAA

The extracellular domain runs from amino acid 51 thought amino acid 229 and is 179 amino acids long with following sequence:

(SEQ ID 3)
GIWSVMQRN CQYVVKQSEL KGTFKGHKCS PCDTNWRYYG
DSCYGFFRHN LTWEESKQYC TDMNATLLKI DNRNIVEYIK
ARTHLIRWVG LSRQKSNEVW KWEDGSVISE NMFEFLEDGK
GNMNCAYFHN GKMHPTFCEN KHYLMCERKA GMTKVDQLP

and is encoded by:

(SEQ ID 4)
GGGATTTGGT CTGTCATGCA GCGCAATTAC CTACAAGGTG
AGAATGAAAA TCGCACAGGA ACTCTGCAAC AATTAGCAAA
GCGCTTCTGT CAATATGTGG TAAAACAATC AGAACTAAAG
GGCACTTTCA AAGGTCATAA ATGCAGCCCC TGTGACACAA
ACTGGAGATA TTATGGAGAT AGCTGCTATG GGTTCTTCAG
GCACAACTTA ACATGGGAAG AGAGTAAGCA GTACTGCACT
GACATGAATG CTACTCTCCT GAAGATTGAC AACCGGAACA
TTGTGGAGTA CATCAAAGCC AGGACTCATT TAATTCGTTG
GGTCGGATTA TCTCGCCAGA AGTCGAATGA GGTCTGGAAG
TGGGAGGATG GCTCGGTTAT CTCAGAAAAT ATGTTTGAGT
TTTTGGAAGA TGGAAAAGGA AATATGAATT GTGCTTATTT
TCATAATGGG AAAATGCACC CTACCTTCTG TGAGAACAAA
CATTATTTAA TGTGTGAGAG GAAGGCTGGC ATGACCAAGG
TGGACCAACT ACCTTAA

The 196 amino acid sequence of full length human CLEC2 isoform 2 is:
MQDEDGYITLNIKTRKPALISAVMQRNYLQGENENRTGTLQQLAKRFCQYVVKQSELKGT FKGHKCSPCDTNWRYYGDSCYGFFRHNLTWEESKQYCTDMNATLLKIDNRNIVEYIKART HLIRWVGLSRQKSNEVWKWEDGSVISENMFEFLEDGKGNMNCAYFHNGKMHPTFCENKHY LMCERKAGMTKVDQLP (SEQ ID 5) and is encoded by the DNA sequence:

(SEQ ID 6)
ATGCAGGATG AAGATGGATA CATCACCTTA AATATTAAAA
CTCGGAAACC AGCTCTCATC TCCGCTGTCA TGCAGCGCAA
TTACCTACAA GGTGAGAATG AAAATCGCAC AGGAACTCTG
CAACAATTAG CAAAGCGCTT CTGTCAATAT GTGGTAAAAC
AATCAGAACT AAAGGGCACT TTCAAAGGTC ATAAATGCAG
CCCCTGTGAC ACAAACTGGA GATATTATGG AGATAGCTGC
TATGGGTTCT TCAGGCACAA CTTAACATGG GAAGAGAGTA
AGCAGTACTG CACTGACATG AATGCTACTC TCCTGAAGAT
TGACAACCGG AACATTGTGG AGTACATCAA AGCCAGGACT
CATTTAATTC GTTGGGTCGG ATTATCTCGC CAGAAGTCGA
ATGAGGTCTG GAAGTGGGAG GATGGCTCGG TTATCTCAGA
AAATATGTTT GAGTTTTTGG AAGATGGAAA AGGAAATATG
AATTGTGCTT ATTTTCATAA TGGGAAAATG CACCCTACCT
TCTGTGAGAA CAAACATTAT TTAATGTGTG AGAGGAAGGC
TGGCATGACC AAGGTGGACC AACTACCTTA A

The 229 amino acid full length murine iso form 1 is:

(SEQ ID NO 7)
MQDEDGYITL NIKPRKQALS SAEPASSWWR VMALVLLISS
MGLVVGLVAL GIMSVTQQKY LLAEKENLSA TLQQLAKKFC
QELIRQSEIK TKSTFEHKCS PCATKWRYHG DSCYGFFRRN
LTWEESKQYC TEQNATLVKT ASQRTLDYIA ERITSVRWIG
LSRQNSKKDW MWEDSSVLRK NGINLSGNTE ENMNCAYLHN
GKIHPASCKE RHYLICERNA GMTRVDQLL

and is encoded by the DNA sequence:

(SEQ ID 8)
ATGCAGGATG AAGATGGGTA TATCACTTTA AACATCAAGC
CCCGGAAACA AGCTCTCAGC TCAGCGGAAC CTGCCTCTTC
TTGGTGGCGT GTGATGGCTT TAGTTCTGCT GATCTCATCC
ATGGGGCTGG TTGTTGGACT CGTGGCTCTG GGGATCATGT
CGGTCACACA GCAAAAGTAT CTACTGGCGG AGAAGGAAAA
TCTCTCAGCG ACTCTGCAAC AATTGGCCAA GAAATTCTGC
CAAGAGTTGA TTAGACAATC AGAAATTAAG ACAAAGAGCA
CTTTTGAGCA CAAGTGCAGC CCCTGCGCCA CGAAGTGGAG
ATACCATGGA GATAGTTGCT ACGGGTTCTT CAGGCGTAAC
CTAACATGGG AAGAGAGCAA GCAGTATTGC ACTGAGCAGA
ATGCAACACT TGTGAAGACT GCCAGCCAGA GAACCCTGGA
CTACATTGCA GAAAGGATTA CTTCAGTCCG TTGGATTGGA
TTATCACGCC AGAACTCTAA GAAAGACTGG ATGTGGGAGG
ATAGCTCAGT TCTTCGCAAG AACGGGATTA ATCTTTCTGG
GAATACAGAA GAAAACATGA ATTGTGCTTA TCTTCATAAT
GGAAAAATCC ATCCAGCTTC CTGTAAAGAG AGACATTACT
TAATATGTGA GAGAAATGCT GGCATGACAA GAGTGGACCA
ACTGCTTTAA

The 179 amino acid sequence of murine extracellular domain of CLEC2 is:

(SEQ ID 9)
GIMSVTQQKYLLAEKENLSATLQQLAKKFCQELIRQSEIKTKSTFEHKCS
PCATKWRYHGDSCYGFERRNLTWEESKQYCTEQNATLVKTASQRTLDYIA
ERITSVRWIGLSRQNSKKDWMWEDSSVLRKNGINLSGNTEENMNCAYLHN
GKIHPASCKERHYLICERNAGMTRVDQLL

and is encoded by the DNA sequence:

(SEQ ID 10)
GGGATCATGTCGGTCACACAGCAAAAGTATCTACTGGCGGAGAAGGAAAA
TCTCTCAGCGACTCTGCAACAATTGGCCAAGAAATTCTGCCAAGAGTTGA
TTAGACAATCAGAAATTAAGACAAAGAGCACTTTTGAGCACAAGTGCAGC
CCCTGCGCCACGAAGTGGAGATACCATGGAGATAGTTGCTACGGGTTCTT
CAGGCGTAACCTAACATGGGAAGAGAGCAAGCAGTATTGCACTGAGCAGA
ATGCAACACTTGTGAAGACTGCCAGCCAGAGAACCCTGGACTACATTGCA
GAAAGGATTACTTCAGTCCGTTGGATTGGATTATCACGCCAGAACTCTAA
GAAAGACTGGATGTGGGAGGATAGCTCAGTTCTTCGCAAGAACGGGATTA
ATCTTTCTGGGAATACAGAAGAAAACATGAATTGTGCTTATCTTCATAAT
GGAAAAATCCATCCAGCTTCCTGTAAAGAGAGACATTACTTAATATGTGA
GAGAAATGCTGGCATGACAAGAGTGGACCAACTGCTTTAA.

As stated herein, the term “CLEC2 polypeptide” refers to a CLEC2 polypeptide comprising the human amino acid sequences SEQ IDs 1, 3 or 5. The term “CLEC2 polypeptide,” however, also encompasses polypeptides comprising an amino acid sequence that differs from the amino acid sequence of a naturally occurring GDF polypeptide sequence, e.g., SEQ IDs 1, 3 or 5, by one or more amino acids, such that the sequence is at least 85% identical, at least 90% identical, at least 95% identical or at least 98% identical to SEQ IDs 1, 3 or 5. CLEC2 polypeptides can be generated by introducing one or more amino acid substitutions, either conservative or non-conservative and using naturally or non-naturally occurring amino acids, at particular positions of the CLEC2 polypeptide.

A “conservative amino acid substitution” can involve a substitution of a native amino acid residue (i.e., a residue found in a given position of the wild-type CLEC2 polypeptide sequence) with a nonnative residue (i.e., a residue that is not found in a given position of the wild-type CLEC2 polypeptide sequence) such that there is little or no effect on the polarity or charge of the amino acid residue at that position. Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Naturally occurring residues can be divided into classes based on common side chain properties:

(1) hydrophobic: norleucine, Met, Ala, Val, Leu, Ile;

(2) neutral hydrophilic: Cys, Ser, Thr;

(3) acidic: Asp, Glu;

(4) basic: Asn, Gln, His, Lys, Arg;

(5) residues that influence chain orientation: Gly, Pro; and

(6) aromatic: Trp, Tyr, Phe.

Additional groups of amino acids can also be formulated using the principles described in, e.g., Creighton (1984) PROTEINS: STRUCTURE AND MOLECULAR PROPERTIES (2d Ed. 1993), W.H. Freeman and Company. In some instances it can be useful to further characterize substitutions based on two or more of such features (e.g., substitution with a “small polar” residue, such as a Thr residue, can represent a highly conservative substitution in an appropriate context).

Conservative substitutions can involve the exchange of a member of one of these classes for another member of the same class. Non-conservative substitutions can involve the exchange of a member of one of these classes for a member from another class.

Synthetic, rare, or modified amino acid residues having known similar physiochemical properties to those of an above-described grouping can be used as a “conservative” substitute for a particular amino acid residue in a sequence. For example, a D-Arg residue may serve as a substitute for a typical L-Arg residue. It also can be the case that a particular substitution can be described in terms of two or more of the above described classes (e.g., a substitution with a small and hydrophobic residue means substituting one amino acid with a residue(s) that is found in both of the above-described classes or other synthetic, rare, or modified residues that are known in the art to have similar physiochemical properties to such residues meeting both definitions).

Nucleic acid sequences encoding a CLEC2 polypeptide provided herein, including those degenerate to SEQ IDs 2, 4 or 6, and those encoding polypeptide variants of SEQ IDs 1, 3 or 5 form other aspects of the instant disclosure.

### II.B. CLEC2 Vectors

In Order to Express the CLEC2 Nucleic Acid Sequences Provided Herein, the Appropriate coding sequences, e.g., SEQ IDs 2, 4, 6, 8, 10, 11 or 12, can be cloned into a suitable vector and after introduction in a suitable host, the sequence can be expressed to produce the encoded polypeptide according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). The invention also relates to such vectors comprising a nucleic acid sequence according to the invention.

A “vector” refers to a delivery vehicle that (a) promotes the expression of a polypeptide-encoding nucleic acid sequence; (b) promotes the production of the polypeptide therefrom; (c) promotes the transfection/transformation of target cells therewith; (d) promotes the replication of the nucleic acid sequence; (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid. A vector can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

A recombinant expression vector can be designed for expression of a CLEC2 protein in prokaryotic (e.g., E. coli) or eukaryotic cells (e.g., insect cells, using baculovirus expression vectors, yeast cells, or mammalian cells). Representative host cells include those hosts typically used for cloning and expression, including Escherichia coli strains TOP10F′, TOP10, DH10B, DH5a, HB101, W3110, BL21(DE3) and BL21 (DE3)pLysS, BLUESCRIPT (Stratagene), mammalian cell lines CHO, CHO-K1, HEK293, 293-EBNA pIN vectors (Van Heeke & Schuster, J. Biol. Chem. 264: 5503-5509 (1989); pET vectors (Novagen, Madison Wis.). Alternatively, the recombinant expression vector can be transcribed and translated in vitro, for example using T7 promoter regulatory sequences and T7 polymerase and an in vitro translation system. Preferably, the vector contains a promoter upstream of the cloning site containing the nucleic acid sequence encoding the polypeptide. Examples of promoters, which can be switched on and off, include the lac promoter, the T7 promoter, the trc promoter, the tac promoter and the trp promoter.

Thus, provided herein are vectors comprising a nucleic acid sequence encoding CLEC2 that facilitate the expression of recombinant CLEC2. In various embodiments, the vectors comprise an operably linked nucleotide sequence which regulates the expression of CLEC2. A vector can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter, EF1alpha promoter, CAG promoter), effective poly (A) termination sequences, an origin of replication for plasmid product in E. coli, an antibiotic resistance gene as a selectable marker, and/or a convenient cloning site (e.g., a polylinker). Vectors also can comprise an inducible promoter as opposed to a constitutive promoter such as CMV IE. In one aspect, a nucleic acid comprising a sequence encoding a CLEC2 polypeptide which is operatively linked to a tissue specific promoter which promotes expression of the sequence in a metabolically-relevant tissue, such as liver or pancreatic tissue is provided.

### II.C. Host Cells

In another aspect of the instant disclosure, host cells comprising the CLEC2 nucleic acids and vectors disclosed herein are provided. In various embodiments, the vector or nucleic acid is integrated into the host cell genome, which in other embodiments the vector or nucleic acid is extra-chromosomal.

Recombinant cells, such as yeast, bacterial (e.g., E. coli), and mammalian cells (e.g., immortalized mammalian cells) comprising such a nucleic acid, vector, or combinations of either or both thereof are provided. In various embodiments cells comprising a non-integrated nucleic acid, such as a plasmid, cosmid, phagemid, or linear expression element, which comprises a sequence coding for expression of a CLEC2 polypeptide, are provided.

A vector comprising a nucleic acid sequence encoding a CLEC2 polypeptide provided herein can be introduced into a host cell by transformation or by transfection. Methods of transforming a cell with an expression vector are well known.

A CLEC2-encoding nucleic acid can be positioned in and/or delivered to a host cell or host animal via a viral vector. Any suitable viral vector can be used in this capacity. A viral vector can comprise any number of viral polynucleotides, alone or in combination with one or more viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), or an intact virus particle comprising viral nucleic acids and a CLEC2 polypeptide-encoding nucleic acid. A viral particle viral vector can comprise a wild-type viral particle or a modified viral particle. The viral vector can be a vector which requires the presence of another vector or wild-type virus for replication and/or expression (e.g., a viral vector can be a helper-dependent virus), such as an adenoviral vector amplicon. Typically, such viral vectors consist of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors.

### II.D. Isolation of a Clec2 Polypeptide

A CLEC2 polypeptide expressed as described herein can be isolated using standard protein purification methods. A CLEC2 polypeptide can be isolated from a cell in which is it naturally expressed or it can be isolated from a cell that has been engineered to express CLEC2, for example a cell that does not naturally express CLEC2.

Protein purification methods that can be employed to isolate a CLEC2 polypeptide, as well as associated materials and reagents, are known in the art. Exemplary methods of purifying a CLEC2 polypeptide are provided in the Examples herein below. Additional purification methods that may be useful for isolating a CLEC2 polypeptide can be found in references such as Bootcov M R, 1997, Proc. Natl. Acad. Sci. USA 94:11514-9, Fairlie W D, 2000, Gene 254: 67-76.

### III. ANTI-CLEC2 ANTIBODIES

Antibodies of the invention include monoclonal antibodies that bind to the CLEC2 receptor, for example to a polypeptide comprising the amino acid sequence of SEQ ID 1, 3, 5, 7, 9 or a ligand to the CLEC2 receptor or a receptor for CLEC2 extracellular domain. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with a CLEC2 receptor or a ligand of the CLEC2 receptor antigen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds a CLEC2 receptor or a ligand of the CLEC2 receptor. Such hybridoma cell lines, and anti-CLEC2 monoclonal antibodies produced by them, are encompassed by the present invention.

Monoclonal antibodies secreted by a hybridoma cell line can be purified using any useful technique known in the antibody arts. Hybridomas or mAbs may be further screened to identify mAbs with particular properties, such as the ability to block a CLEC2 induced activity.

Chimeric and humanized antibodies based upon the foregoing sequences are also provided by the present invention. Monoclonal antibodies for use as therapeutic agents may be modified in various ways prior to use. One example is a “chimeric” antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., Proc. Natl. Acad. Sci. USA 81:6851-6855 (1985). CDR grafting is described, for example, in U.S. Pat. Nos. 6,180,370, 5,693,762, 5,693,761, 5,585,089, and 5,530,101.

Generally, the goal of making a chimeric antibody is to create a chimera in which the number of amino acids from the intended patient species is maximized. One example is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the V region or selected CDRs from a rodent antibody often are grafted into a human antibody, replacing the naturally-occurring V regions or CDRs of the human antibody.

One useful type of chimeric antibody provided by the present invention is a “humanized” antibody. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089, and 5,693,762; Jones et al., 1986, Nature 321:522-25; Riechmann et al., 1988, Nature 332:323-27; Verhoeyen et al., 1988, Science 239:1534-36). In certain embodiments, constant regions from species other than human can be used along with the human variable region(s) to produce hybrid antibodies.

Fully human antibodies are also provided. Methods are available for making fully human antibodies specific for a given antigen without exposing human beings to the antigen (“fully human antibodies”). One means for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (MAbs) in mouse, an animal that can be immunized with any desirable antigen. Using fully human antibodies can minimize the immunogenic and allergic responses that can sometimes be caused by administering mouse or mouse-derivatized Mabs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,545,807; 6,300,129; 6,255,458; 5,877,397; 5,874,299 and 5,545,806; in PCT publications WO91/10741, WO90/04036, and in EP 546073B1 and EP 546073A1.

The transgenic mice described above, referred to herein as “HuMab” mice, contain a human immunoglobulin gene minilocus that encodes unrearranged human heavy μ and gamma) and kappa light chain immunoglobulin sequences, together with targeted mutations that inactivate the endogenous μ and .kappa. chain loci (Lonberg et al., 1994, Nature 368: 856-859).

Accordingly, the aforementioned mice exhibit reduced expression of mouse IgM or kappa and in response to immunization, and the introduced human heavy and light chain transgenes undergo class switching and somatic mutation to generate high affinity human IgG kappa monoclonal antibodies (Lonberg et al., supra.; Lonberg and Huszar, 1995, Intern. Rev. Immunol., 13: 65-93; Harding and Lonberg, 1995, Ann. N.Y Acad. Sci 764: 536-546). The preparation of HuMab mice is described in detail in Taylor et al., 1992, Nucleic Acids Research, 20: 6287-6295; Chen et al., 1993, International Immunology 5: 647-656; Tuaillon et al., 1994, J. Immunol. 152: 2912-2920; Lonberg et al., 1994, Nature 368: 856-859; Lonberg, 1994, Handbook of Exp. Pharmacology 113: 49-101; Taylor et al., 1994, International Immunology 6: 579-591; Lonberg and Huszar, 1995, Intern. Rev. Immunol. 13: 65-93; Harding and Lonberg, 1995, Ann. N.Y Acad. Sci. 764: 536-546; Fishwild et al., 1996, Nature Biotechnology 14: 845-851. See further U.S. Pat. Nos. 5,545,806; 5,569,825; 5,625,126; 5,633,425; 5,789,650; 5,877,397; 5,661,016; 5,814,318; 5,874,299; and 5,770,429; as well as U.S. Pat. No. 5,545,807; International Publication Nos. WO 93/1227; WO 92/22646; and WO 92/03918. Technologies utilized for producing human antibodies in these transgenic mice are disclosed also in WO 98/24893, and Mendez et al., 1997, Nature Genetics 15: 146-156.

Using hybridoma technology, antigen-specific human MAbs with the desired specificity can be produced and selected from the transgenic mice such as those described above. Such antibodies may be cloned and expressed using a suitable vector and host cells.

Fully human antibodies of the invention can also be derived from phage-display libraries (as disclosed in Hoogenboom et al., 1991, J. Mol. Biol. 227:381; and Marks et al., 1991, J. Mol. Biol. 222:581). Phage display techniques mimic immune selection through the display of antibody repertoires on the surface of filamentous bacteriophage, and subsequent selection of phage by their binding to an antigen of choice. One such technique is described in PCT Publication No. WO99/10494, which describes the isolation of high affinity and functional agonistic antibodies for MPL- and msk-receptors using such an approach.

The anti-CLEC2 agents provided herein may also block or reduce binding between the CLEC2 receptor and a ligand thereby inhibiting signaling through the CLEC2 pathway. The agents can be an antibody or an immunologically functional fragment thereof and thus include antibodies with a naturally occurring structure, as well as polypeptides that have an antigen binding domain (e.g., a domain antibody). The antibodies and fragments can be used to treat a variety of metabolic conditions. Nucleic acids molecules, vectors, and host cells useful in the production of the antibodies are also provided.

Also included are isolated antibodies or an immunologically functional fragments thereof that specifically bind a CLEC2 polypeptide comprising an amino acid sequence of SEQ ID 1, 3 or 5 or to a polypeptide at least 90%, 95%, 98% identical thereto, respectively.

### IV. PHARMACEUTICAL COMPOSITIONS COMPRISING A CLEC2 POLYPEPTIDE OR CLEC2 ANTIBODY

Pharmaceutical compositions comprising a CLEC2 polypeptide or anti-CLEC2 antibody are provided. Such CLEC2 polypeptide or anti-CLEC2 antibody pharmaceutical compositions can comprise a therapeutically effective amount of a CLEC2 polypeptide or anti-CLEC2 antibody in admixture with a pharmaceutically or physiologically acceptable formulation agent selected for suitability with the mode of administration. The term “pharmaceutically acceptable carrier” or “physiologically acceptable carrier” as used herein refers to one or more formulation agents suitable for accomplishing or enhancing the delivery of a CLEC2 polypeptide or anti-CLEC2 antibody into the body of a human or non-human subject. The term includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In some cases it will be preferable to include isotonic agents, for example, sugars, polyalcohols such as mannitol, sorbitol, or sodium chloride in a pharmaceutical composition. Pharmaceutically acceptable substances such as wetting or minor amounts of auxiliary substances such as wetting or emulsifying agents, preservatives or buffers, which enhance the shelf life or effectiveness of the CLEC2 polypeptide or anti-CLEC2 antibody can also act as, or form a component of, a carrier. Acceptable pharmaceutically acceptable carriers are preferably nontoxic to recipients at the dosages and concentrations employed.

A pharmaceutical composition can contain formulation agent(s) for modifying, maintaining, or preserving, for example, the pH, osmolarity, viscosity, clarity, color, isotonicity, odor, sterility, stability, rate of dissolution or release, adsorption, or penetration of the composition. Suitable formulation agents include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON: THE SCIENCE AND PRACTICE OF PHARMACY, 19th edition, (1995); Berge et al., J. Pharm. Sci., 6661), 1-19 (1977). Additional relevant principles, methods, and agents are described in, e.g., Lieberman et al., PHARMACEUTICAL DOSAGE FORMS-DISPERSE SYSTEMS (2nd ed., vol. 3, 1998); Ansel et al., PHARMACEUTICAL DOSAGE FORMS & DRUG DELIVERY SYSTEMS (7th ed. 2000); Martindale, THE EXTRA PHARMACOPEIA (31st edition), Remington's PHARMACEUTICAL SCIENCES (16th-20^{th} and subsequent editions); The Pharmacological Basis Of Therapeutics, Goodman and Gilman, Eds. (9th ed.—1996); Wilson and Gisvolds' TEXTBOOK OF ORGANIC MEDICINAL AND PHARMACEUTICAL CHEMISTRY, Delgado and Remers, Eds. (10th ed., 1998). Principles of formulating pharmaceutically acceptable compositions also are described in, e.g., Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), incorporated herein by reference for any purpose).

The optimal pharmaceutical composition will be determined by a skilled artisan depending upon, for example, the intended route of administration, delivery format, and desired dosage (see, e.g., Remington's PHARMACEUTICAL SCIENCES, supra). Such compositions can influence the physical state, stability, rate of in vivo release, and rate of in vivo clearance of the a CLEC2 polypeptide.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the present invention, FGF21 polypeptide mutant compositions can be prepared for storage by mixing the selected composition having the desired degree of purity with optional formulation agents (Remington's PHARMACEUTICAL SCIENCES, supra) in the form of a lyophilized cake or an aqueous solution. Furthermore, the CLEC2 polypeptide or anti-CLEC2 antibody product can be formulated as a lyophilizate using appropriate excipients such as sucrose.

The CLEC2 polypeptide or anti-CLEC2 antibody pharmaceutical compositions can be selected for parenteral delivery. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention can be in the form of a pyrogen-free, parenterally acceptable, aqueous solution comprising the desired CLEC2 polypeptide in a pharmaceutically acceptable vehicle. A particularly suitable vehicle for parenteral injection is sterile distilled water in which a CLEC2 polypeptide or anti-CLEC2 antibody is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a CLEC2 inhibitor such as a CLEC2 polypeptide or anti-CLEC2 antibody can be formulated as a dry powder for inhalation. A CLEC2 polypeptide or anti-CLEC2 antibody inhalation solutions can also be formulated with a propellant for aerosol delivery. In yet another embodiment, solutions can be nebulized. Pulmonary administration is further described in International Publication No. WO 94/20069, which describes the pulmonary delivery of chemically modified proteins.

It is also contemplated that certain formulations can be administered orally. In one embodiment of the present invention, the CLEC2 polypeptide or anti-CLEC2 antibody that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the CLEC2 polypeptide or anti-CLEC2 antibody. Diluents, flavorings, low melting point waxes, vegetable oils, lubricants, suspending agents, tablet disintegrating agents, and binders can also be employed.

Another pharmaceutical composition can involve an effective quantity of a CLEC2 polypeptide or anti-CLEC2 antibody in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional CLEC2 polypeptide pharmaceutical compositions will be evident to those skilled in the art, including formulations involving a CLEC2 polypeptide or anti-CLEC2 antibody in sustained- or controlled-delivery formulations. Techniques for formulating a variety of other sustained- or controlled-delivery means, such as liposome carriers, bio-erodible microparticles or porous beads and depot injections, are also known to those skilled in the art (see, e.g., International Publication No. WO 93/15722, which describes the controlled release of porous polymeric microparticles for the delivery of pharmaceutical compositions, and Wischke & Schwendeman, 2008, Int. J. Pharm. 364: 298-327, and Freiberg & Zhu, 2004, Int. J. Pharm. 282: 1-18, which discuss microsphere/microparticle preparation and use). As described herein, a hydrogel is an example of a sustained- or controlled-delivery formulation.

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et al., 1983, Biopolymers 22: 547-56), poly(2-hydroxyethyl-methacrylate) (Langer et al., 1981, J. Biomed. Mater. Res. 15: 167-277 and Langer, 1982, Chem. Tech. 12: 98-105), ethylene vinyl acetate (Langer et al., supra) or poly-D(−)-3-hydroxybutyric acid (European Patent No. 0 133 988). Sustained-release compositions can also include liposomes, which can be prepared by any of several methods known in the art. See, e.g., Epstein et al., 1985, Proc. Natl. Acad. Sci. U.S.A. 82: 3688-92; and European Patent Nos. 0 036 676, 0 088 046, and 0 143 949.

A CLEC2 polypeptide or anti-CLEC2 antibody pharmaceutical composition to be used for in vivo administration typically should be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the present invention is directed to kits for producing a single dose administration unit. The kits can each contain both a first container having a dried protein and a second container having an aqueous formulation. Also included within the scope of this invention are kits containing single and multi-chambered pre-filled syringes (e.g., liquid syringes and lyosyringes).

The effective amount of a CLEC2 polypeptide or anti-CLEC2 antibody pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which a CLEC2 polypeptide or anti-CLEC2 antibody is being used, the route of administration, and the size (body weight, body surface, or organ size) and condition (the age and general health) of the patient. Accordingly, the clinician can titer the dosage and modify the route of administration to obtain the optimal therapeutic effect. A typical dosage can range from about 0.1 μg/kg to up to about 100 mg/kg or more, depending on the factors mentioned above.

The frequency of dosing will depend upon the pharmacokinetic parameters of the CLEC2 polypeptide or anti-CLEC2 antibody, in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems (which may also be injected); or by implantation devices. Where desired, the compositions can be administered by bolus injection or continuously by infusion, or by implantation device.

Alternatively or additionally, the composition can be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

In order to deliver drug, e.g., a CLEC2 polypeptide or anti-CLEC2 antibody, at a predetermined rate such that the drug concentration can be maintained at a desired therapeutically effective level over an extended period, a variety of different approaches can be employed. In one example, a hydrogel comprising a polymer such as a gelatin (e.g., bovine gelatin, human gelatin, or gelatin from another source) or a naturally-occurring or a synthetically generated polymer can be employed. Any percentage of polymer (e.g., gelatin) can be employed in a hydrogel, such as 5, 10, 15 or 20%. The selection of an appropriate concentration can depend on a variety of factors, such as the therapeutic profile desired and the pharmacokinetic profile of the therapeutic molecule.

Examples of polymers that can be incorporated into a hydrogel include polyethylene glycol (“PEG”), polyethylene oxide, polyethylene oxide-co-polypropylene oxide, co-polyethylene oxide block or random copolymers, polyvinyl alcohol, poly(vinyl pyrrolidinone), poly(amino acids), dextran, heparin, polysaccharides, polyethers and the like.

Another factor that can be considered when generating a hydrogel formulation is the degree of crosslinking in the hydrogel and the crosslinking agent. In one embodiment, cross-linking can be achieved via a methacrylation reaction involving methacrylic anhydride. In some situations, a high degree of cross-linking may be desirable while in other situations a lower degree of crosslinking is preferred. In some cases a higher degree of crosslinking provides a longer sustained release. A higher degree of crosslinking may provide a firmer hydrogel and a longer period over which drug is delivered.

Any ratio of polymer to crosslinking agent (e.g., methacrylic anhydride) can be employed to generate a hydrogel with desired properties. For example, the ratio of polymer to crosslinker can be, e.g., 8:1, 16:1, 24:1, or 32:1. For example, when the hydrogel polymer is gelatin and the crosslinker is methacrylate, ratios of 8:1, 16:1, 24:1, or 32:1 methyacrylic anhydride:gelatin can be employed.

### V. THERAPEUTIC USES OF CLEC2 POLYPEPTIDES AND ANTIBODIES

A CLEC2 polypeptide or anti-CLEC2 antibody, can be used to treat, diagnose or ameliorate, a metabolic condition or disorder. In one embodiment, the metabolic disorder to be treated is diabetes, e.g., type 2 diabetes. In another embodiment, the metabolic condition or disorder is obesity. In other embodiments the metabolic condition or disorder is elevated glucose levels, elevated insulin levels, elevated triglyceride levels or poor glucose tolerance or insulin insensitivity. For example, a metabolic condition or disorder that can be treated or ameliorated using a CLEC2 polypeptide or anti-CLEC2 antibody, includes a state in which a human subject has a fasting blood glucose level of 125 mg/dL or greater, for example 130, 135, 140, 145, 150, 155, 160, 165, 170, 175, 180, 185, 190, 195, 200 or greater than 200 mg/dL. Blood glucose levels can be determined in the fed or fasted state, or at random. The metabolic condition or disorder can also comprise a condition in which a subject is at increased risk of developing a metabolic condition. For a human subject, such conditions include a fasting blood glucose level of 100 mg/dL. Conditions that can be treated using a pharmaceutical composition comprising a CLEC2 polypeptide or anti-CLEC2 antibody, can also be found in the American Diabetes Association Standards of Medical Care in Diabetes Care-2011, American Diabetes Association, Diabetes Care Vol. 34, No. Supplement 1, S11-S61, 2010, incorporated herein by reference.

In application, a metabolic disorder or condition, such as Type 2 diabetes, elevated glucose levels, elevated insulin levels, insulin resistance and poor glucose tolerance can be treated by administering a therapeutically effective dose of an anti-CLEC2 antibody or a CLEC2 polypeptide, e.g., a human CLEC2 polypeptide such as SEQ IDs 1, 3, 5 or 12, to a patient in need thereof. The administration can be performed as described herein, such as by IV injection, intraperitoneal (IP) injection, subcutaneous injection, intramuscular injection, or orally in the form of a tablet or liquid formation. In some situations, a therapeutically effective or preferred dose of a CLEC2 polypeptide or anti-CLEC2 antibody, can be determined by a clinician. A therapeutically effective dose of a CLEC2 polypeptide or anti-CLEC2 antibody, will depend, inter alia, upon the administration schedule, the unit dose of agent administered, whether the CLEC2 polypeptide or anti-CLEC2 antibody, is administered in combination with other therapeutic agents, the immune status and the health of the recipient. The term “therapeutically effective dose,” as used herein, means an amount of a CLEC2 polypeptide or anti-CLEC2 antibody, that elicits a biological or medicinal response in a tissue system, animal, or human being sought by a researcher, medical doctor, or other clinician, which includes alleviation or amelioration of the symptoms of the disease or disorder being treated, i.e., an amount of a CLEC2 polypeptide or anti-CLEC2 antibody, that supports an observable level of one or more desired biological or medicinal response, for example lowering blood glucose, insulin, triglyceride, or cholesterol levels; reducing body weight; or improving glucose tolerance, energy expenditure, or insulin sensitivity.

It is noted that a therapeutically effective dose of CLEC2 polypeptide or anti-CLEC2 antibody, can also vary with the desired result. Thus, for example, in situations in which a lower level of blood glucose is indicated a dose of a CLEC2 polypeptide or anti-CLEC2 antibody, will be correspondingly higher than a dose in which a comparatively lower level of blood glucose is desired. Conversely, in situations in which a higher level of blood glucose is indicated a dose of a CLEC2 polypeptide or anti-CLEC2 antibody will be correspondingly lower than a dose in which a comparatively higher level of blood glucose is desired.

In various embodiments, a subject is a human having a blood glucose level of 100 mg/dL or greater can be treated with a CLEC2 polypeptide or anti-CLEC2 antibody,

In one embodiment, a method of the instant disclosure comprises first measuring a baseline level of one or more metabolically-relevant compounds such as glucose, insulin, triglyceride level in a subject. A pharmaceutical composition comprising a CLEC2 polypeptide or anti-CLEC2 antibody, is then administered to the subject. After a desired period of time, the level of the one or more metabolically-relevant compounds (e.g., blood glucose, insulin, triglyceride) in the subject is again measured. The two levels can then be compared in order to determine the relative change in the metabolically-relevant compound in the subject. Depending on the outcome of that comparison another dose of the pharmaceutical composition comprising a CLEC2 polypeptide or anti-CLEC2 antibody, molecule can be administered to achieve a desired level of one or more metabolically-relevant compound.

It is noted that a pharmaceutical composition comprising a CLEC2 polypeptide or anti-CLEC2 antibody, can be co-administered with another compound. The identity and properties of compound co-administered with the CLEC2 polypeptide or anti-CLEC2 antibody, will depend on the nature of the condition to be treated or ameliorated. A non-limiting list of examples of compounds that can be administered in combination with a pharmaceutical composition comprising a CLEC2 polypeptide or anti-CLEC2 antibody, include rosiglitizone, pioglitizone, repaglinide, nateglitinide, metformin, exenatide, stiagliptin, pramlintide, glipizide, glimeprirideacarbose, and miglitol.

### VI. KITS

Also provided are kits for practicing the disclosed methods. Such kits can comprise a pharmaceutical composition such as those described herein, including nucleic acids encoding the peptides or proteins provided herein, vectors and cells comprising such nucleic acids, and pharmaceutical compositions comprising such nucleic acid-containing compounds, which can be provided in a sterile container. Optionally, instructions on how to employ the provided pharmaceutical composition in the treatment of a metabolic disorder can also be included or be made available to a patient or a medical service provider.

In one aspect, a kit comprises (a) a pharmaceutical composition comprising a therapeutically effective amount of a CLEC2 polypeptide or anti-CLEC2 antibody, and (b) one or more containers for the pharmaceutical composition. Such a kit can also comprise instructions for the use thereof; the instructions can be tailored to the precise metabolic disorder being treated. The instructions can describe the use and nature of the materials provided in the kit. In certain embodiments, kits include instructions for a patient to carry out administration to treat a metabolic disorder, such as elevated glucose levels, elevated insulin levels, elevated triglyceride level, poor glucose tolerance, poor insulin sensitivity and/or type 2 diabetes.

Instructions can be printed on a substrate, such as paper or plastic, etc, and can be present in the kits as a package insert, in the labeling of the container of the kit or components thereof (e.g., associated with the packaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, such as over the internet, are provided. An example of this embodiment is a kit that includes a web address where the instructions can be viewed and/or from which the instructions can be downloaded.

Often it will be desirable that some or all components of a kit are packaged in suitable packaging to maintain sterility. The components of a kit can be packaged in a kit containment element to make a single, easily handled unit, where the kit containment element, e.g., box or analogous structure, may or may not be an airtight container, e.g., to further preserve the sterility of some or all of the components of the kit.

### EXAMPLES

The following examples, including the experiments conducted and results achieved, are provided for illustrative purposes only and are not to be construed as limiting the present invention.

### Example 1

### Preparation of CLEC2 Fusion Protein Inhibitor

The hFc-CLEC2 (ECD) expression vector comprises amino acids 51-229 of mouse CLEC2 protein, fused at the N-terminal with the Fc portion of human IgG1. A short 11 amino acid long GS linker was also inserted between human Fc and mouse CLEC2 to result in the hFc-CLEC2 (ECD) cDNA having the following sequence:

SEQ ID 11
ATGGAATGGAGCTGGGTCTTTCTCTTCTTCCTGTCAGTAACTACAGGTGT
CCACTCCGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAGCCACAGGTGTACACCCTG
CCCCCATCCCGGGATGAGCTGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCTCCGGGTAAAGGAGGCGGTGGA
TCTGGCGGAGGTGGAGCTAGCGGGATCATGTCGGTCACACAGCAAAAGTA
TCTACTGGCGGAGAAGGAAAATCTCTCAGCGACTCTGCAACAATTGGCCA
AGAAATTCTGCCAAGAGTTGATTAGACAATCAGAAATTAAGACAAAGAGC
ACTTTTGAGCACAAGTGCAGCCCCTGCGCCACGAAGTGGAGATACCATGG
AGATAGTTGCTACGGGTTCTTCAGGCGTAACCTAACATGGGAAGAGAGCA
AGCAGTATTGCACTGAGCAGAATGCAACACTTGTGAAGACTGCCAGCCAG
AGAACCCTGGACTACATTGCAGAAAGGATTACTTCAGTCCGTTGGATTGG
ATTATCACGCCAGAACTCTAAGAAAGACTGGATGTGGGAGGATAGCTCAG
TTCTTCGCAAGAACGGGATTAATCTTTCTGGGAATACAGAAGAAAACATG
AATTGTGCTTATCTTCATAATGGAAAAATCCATCCAGCTTCCTGTAAAGA
GAGACATTACTTAATATGTGAGAGAAATGCTGGCATGACAAGAGTGGACC
AACTGCTTTAA

The entire hFc-CLEC2 (ECD) cDNA (SEQ ID 11) was cloned into pTT5, a CMV based mammalian expression vector.

Large scale transient transfection was used to produce enough conditioned media for protein purification. Up-to 10 mgs of endotoxin-free expression vector plasmid DNA was transfected into 10 liters of 293ENBA (293E) cells along with desired transfection reagent Transfected 293E cells were incubated at 37 degree in a humidified incubator with 5% CO2 concentration. At day 7-9 post transfection, the 293E cell culture was harvested by centrifugation and the supernatant was collected for protein purification.

Clarified conditioned cell culture medium containing Fc-CLEC2 protein was concentrated up to 10 fold by diafiltration on a 10K regenerated cellulose membrane against DPBS. Retentate was clarified and applied to an appropriately sized MabSelect Sure Protein A affinity column with a 2-3 minute residence time, washed in DPBS, and eluted at pH 3.5. Affinity purified Fc-CLEC2 protein was either resolved on an appropriately sized SuperDex200 size exclusion column pre-equilibrated in 0.15M NaCl buffered at neutral pH or the hFc-CLEC2 protein A eluate was adjusted to neutral pH and applied to a sulphopropyl cation exchange column at up to 15 mg/ml resin with a 5-8 minute residence time and resolved in a NaCl gradient buffered at neutral pH. By either method, host cell contaminants, aggregate, and endotoxins were removed and fractions were pooled, reconcentrated by ultrafiltration, sterile filtered and stored frozen.

### Example 2

### AAV Expression of Murine CLEC-2 Extracellular Domain Fusion Protein Reduces Blood Insulin Levels, Blood Glucose Levels and Improves Oral Glucose Tolerance in a Murine Diet Induced Obesity Model

Recombinant adeno-associated virus (rAAV) vector was used to achieve in vivo over expression in a DIO mouse model. As described in Example 1, the murine C-terminal extracellular domain of Clec-2 was fused to the C-terminus of a human Fc, resulting in hFc-mCLEC2 (ECD) (SEQ ID 11) which was cloned into AAV vector with an EF1a promoter and bGH polyA. rAAVs were produced by transient transfection into 293T cells using helper-free system, purified by gradient centrifugation, buffer exchanged, and concentrated. Purified rAAVs were titrated by a fluorescence-method.

12-week-old male B6D2F1 DIO mice (from Jackson Lab) were housed in 4 to 5 groups. The mice were fed a high fat diet (up to 60% fat) for a period of up to 20 weeks. The baseline metabolic profile of body weight, glucose and insulin level were measured by collecting blood from the tail tip after 0 to 16 hours fasting.

Mice were divided into two groups (n=12) based on body weight and baseline glucose. Group 1 was injected, through the tail vein with injected with 8×10¹² genomic copy/animal of control AAV which was the empty vector that did not comprise hFc-mCLEC2. Group 2 was injected, through the tail vein, with 8×10¹² genomic copy/animal of AAV expressing hFc-mCLEC2.

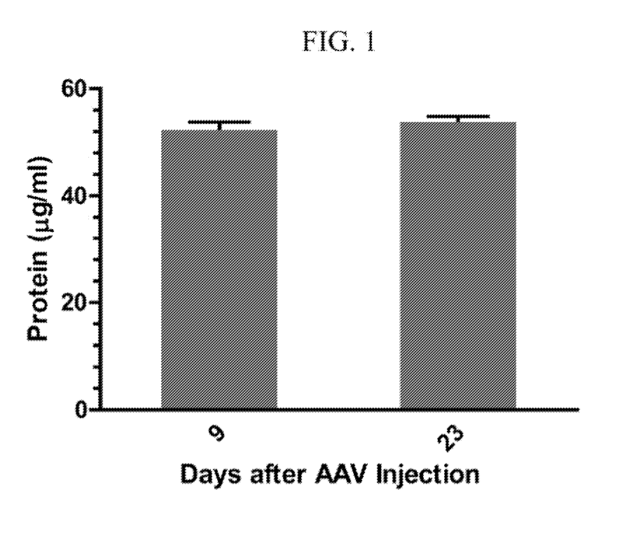

Serum protein levels of hFc-mCLEC2(ECD) was measured by hFc Elisa 9 days and 23 days post injection (FIG. 1) as follows: Maxisorp plates were coated with 1.0 μg/ml of goat anti-human Fc in PBS over night at 4° C., and washed one time with PBS. The plate was blocked with 3% BSA in PBS over night at 4° C., and washed one time with PBS. Serum samples at appropriate dilution in PBS+1% BSA were added and incubated for 1 h at room temperature and washed 3 times with PBS+0.01% Tween-20. Goat anti-human Fc-HRP in PBS+1% BSA, was added and incubated for 1 h at room temperature. The plate was washed 6 times with PBS+0.01% Tween-20. Substrate TMB was added and incubated for 2-30 min. 1N HCl was added to stop the reactions. The plates were read on a SpecMax plate reader at 450 nm. Recombinant human Fc protein was used for standard curve.

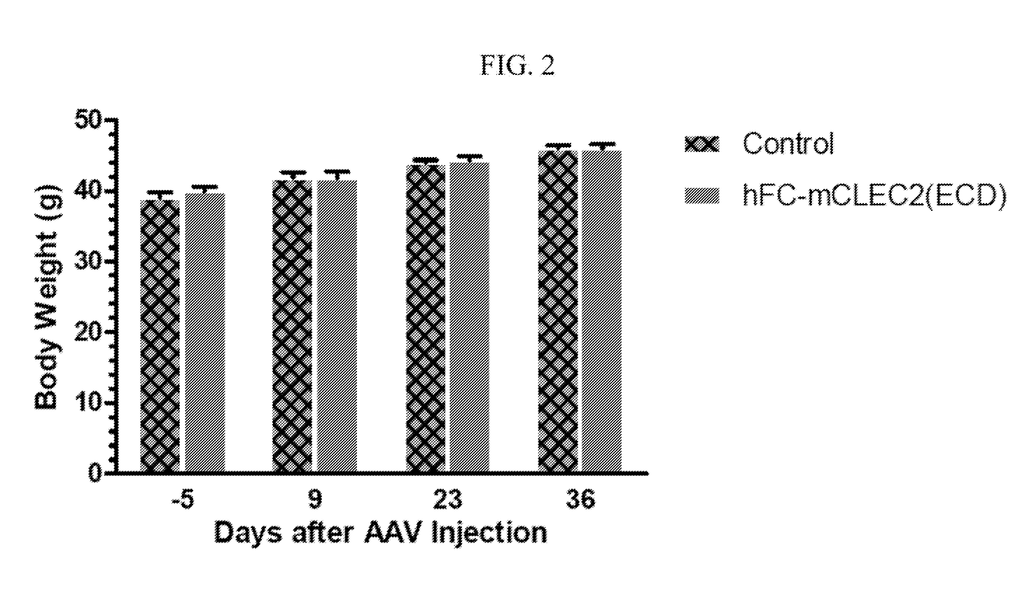

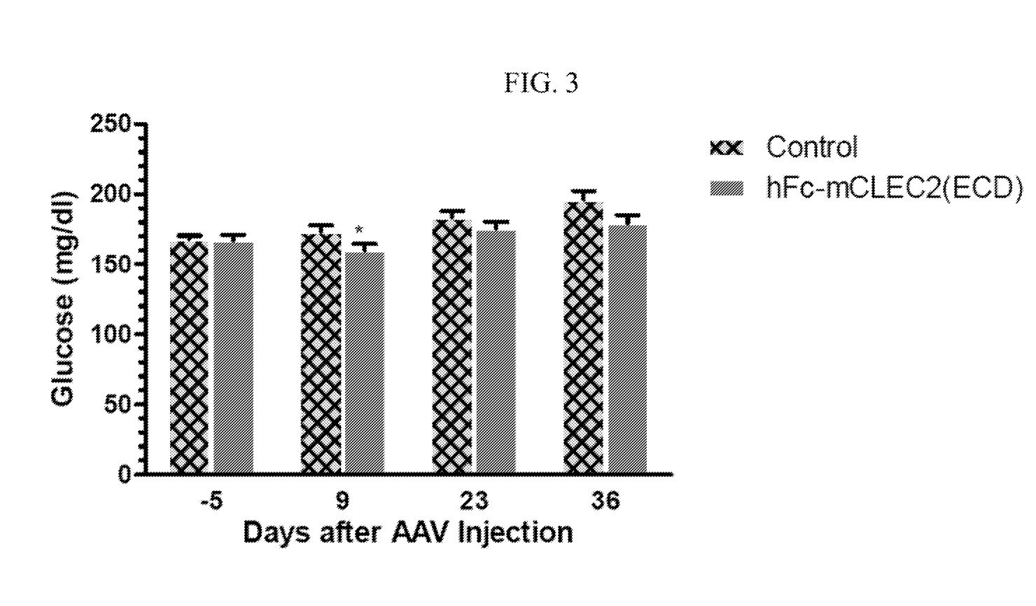

Body weight and glucose levels were measured five days before injection and on days 9, 23 and 36 after injection (FIGS. 2 and 3 respectively).

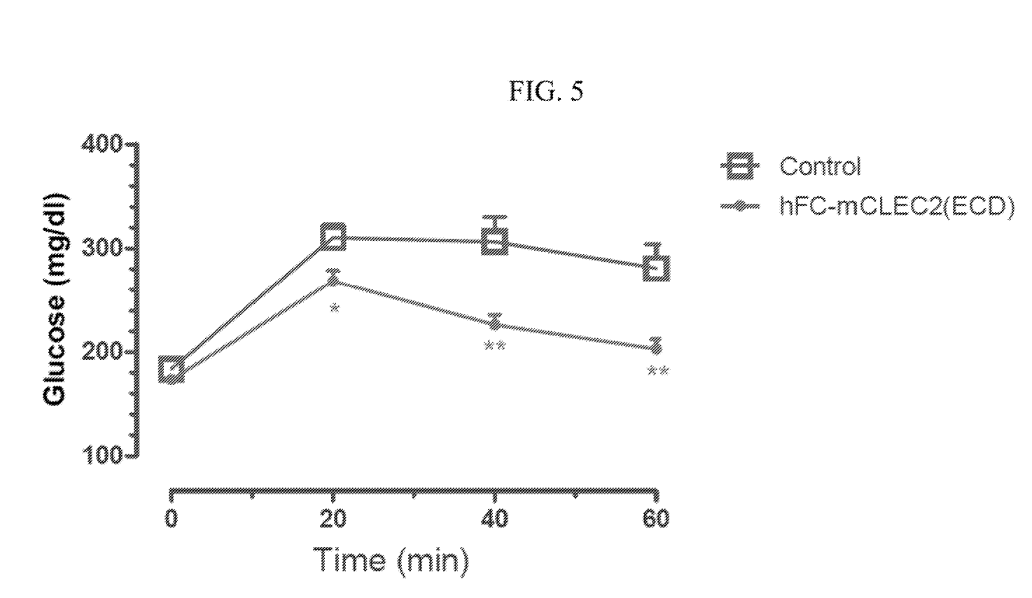

Oral glucose tolerance was measured 23 days post injection as follows: Animals were fasted for 4 hours. Following measurement of body weight and glucose levels (by glucometer), mice were injected with a bolus of glucose (10 ml/kg body weight of 20% glucose) into the stomach by a gavage needle. Glucose levels were measured with a glucometer by tail tip blood collection at 0, 20, 40 and 60 min after glucose dosing. FIG. 5 shows the glucose curve. The hFc-mCLEC2 (ECD) group had lower glucose levels at all time points compared to the control group, indicating that hFc-mCLEC2 (ECD) treated animals have improved glucose tolerance.

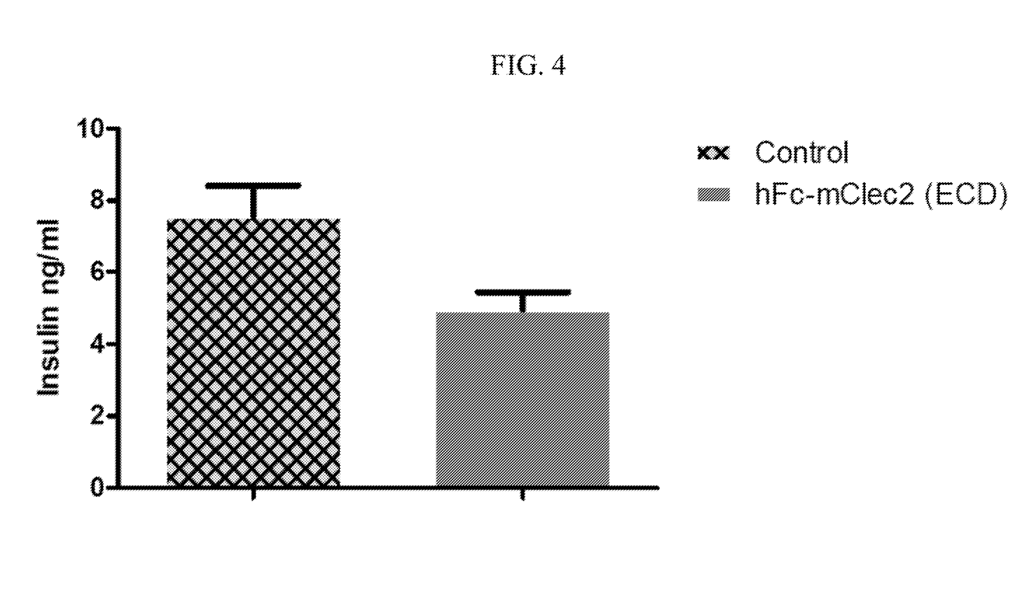

Plasma insulin level was measured on day 40 following injection (FIG. 4) using the mouse Insulin ELISA kit from ALPCO Diagnostics (catalog number 80-INSMS-E01). Samples were diluted 1:10 in zero standard and 10 ul of diluted sample was used per well. The assay was performed as described in the manufacturer's protocol.

The lowered blood glucose and insulin levels and improved oral glucose tolerance in the hFc-mCLEC2(ECD) AAV-group compared to control virus treated group demonstrated that AAV mediated in vivo overexpression of hFc-mCLEC2(ECD), largely corrected metabolic abnormalities in DIO mice, including hyperglycemia and, hyperinsulinemia, insulin resistance. This data confirmed our hypothesis that CLEC2 is involved in the regulation of body metabolism and that antagonism of CLEC2 can treat metabolic disorders, particularly diabetes.

### Example 3

### HTV Injection of Murine CLEC-2 Extracellular Domain Fusion Protein Reduces Blood Insulin Levels, Blood Glucose Levels, Liver Triglyceride Levels and Improves Oral Glucose Tolerance in a Murine Diet Induced Obesity Model

Hydrodynamic tail vein injection (“HTV”) was used to achieve in vivo over expression in a DIO mouse model. The murine C-terminal extracellular domain of Clec-2 was fused to a human F_{c}, resulting in hFc-mCLEC2 (ECD)(SEQ ID 11) which was cloned into the HTV construct vector with an UBC6 promoter and bGHpolyA.

12-week-old male B6D2F1 DIO mice (from Jackson Lab) were housed in 4 to 5 groups. The mice were fed a high fat diet (up to 60% fat) for a period of up to 20 weeks. The baseline metabolic profile of body weight, glucose and insulin level were measured by collecting blood from the tail tip after 0 to 16 hours fasting.

Mice were divided into two groups (n=15) based on body weight and baseline glucose. Group 1 was injected, through the tail vein, with 10 ug per animal of the control HTV DNA construct in a volume of 2.5 ml. The control HTV DNA construct expressed human Fc only. Group 2 was injected, through the tail vein, with 20 ug per animal of the HTV DNA construct expressing hFc-mCLEC2 in a volume of 2.5 ml. Injections of both constructs were preformed as follows: Briefly, the control or hFc-mCLEC2 DNA construct initially prepared at 20 ug/ml, having a plasmid preparation endotoxin level of 100 EU/mg of DNA, was diluted in saline or Ringer's solution. A volume up to 100 mL/kg (10%) of solution, not to exceed 2.5 ml was injected into the tail vein of mice within a 5-8 second timeframe.

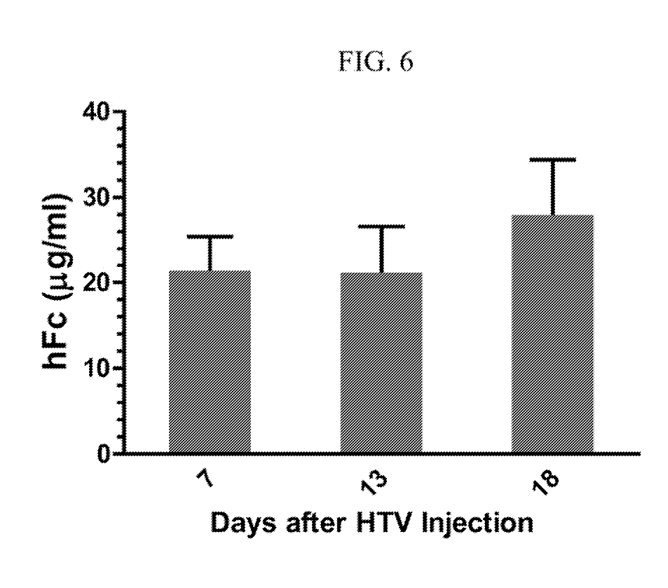

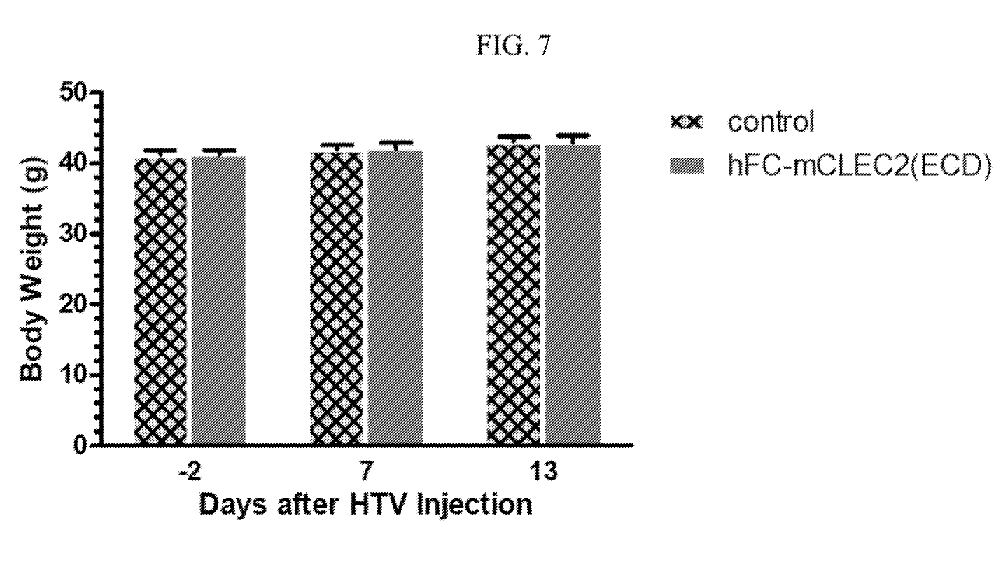

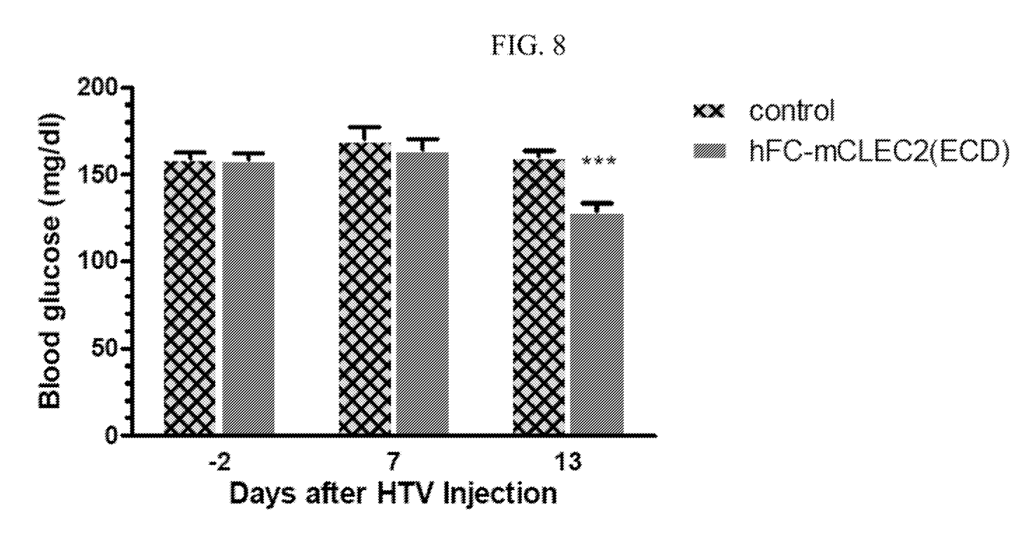

Serum protein levels of hFc-mCLEC2(ECD) were measured by hFc ELISA 7, 13 and 18 days post injection (FIG. 6) using the hFc ELISA described in Example 2 above. Body weight and blood glucose levels were measured 2 days before injection and on days 7 and 13 after injection (FIGS. 7 and 8 respectively).

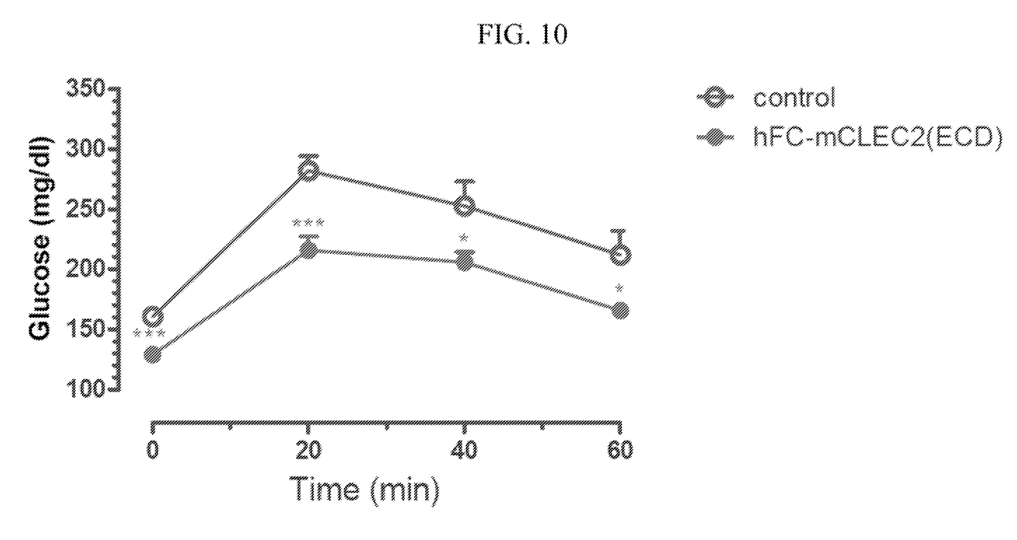

Oral glucose tolerance was measured 13 days post injection The OGTT was performed as follows: animals were fasted for 4 hours. Following measurement of body weight and glucose levels (by glucometer), mice were injected with a bolus of glucose (10 ml/kg body weight of 20% glucose) into the stomach by a gavage needle. Glucose levels were measured with a glucometer by tail tip blood collection at 0, 20, 40 and 60 min after glucose dosing. FIG. 10 shows that hFc-mCLEC2(ECD) treated animals had lower glucose at every time point indicating improved glucose tolerance.

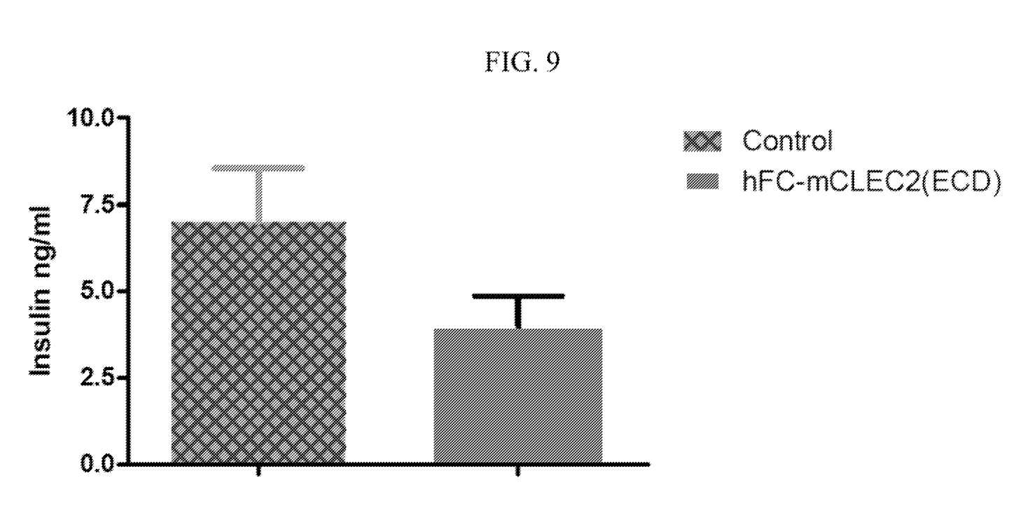

Plasma insulin levels were measured on day 18 following injection (FIG. 9) using the mouse Insulin ELISA kit from ALPCO Diagnostics (catalog number 80-INSMS-E01). Samples were diluted 1:10 in zero standard and 10 ul of diluted sample was used per well. The assay was performed as described in the manufacturer's protocol.

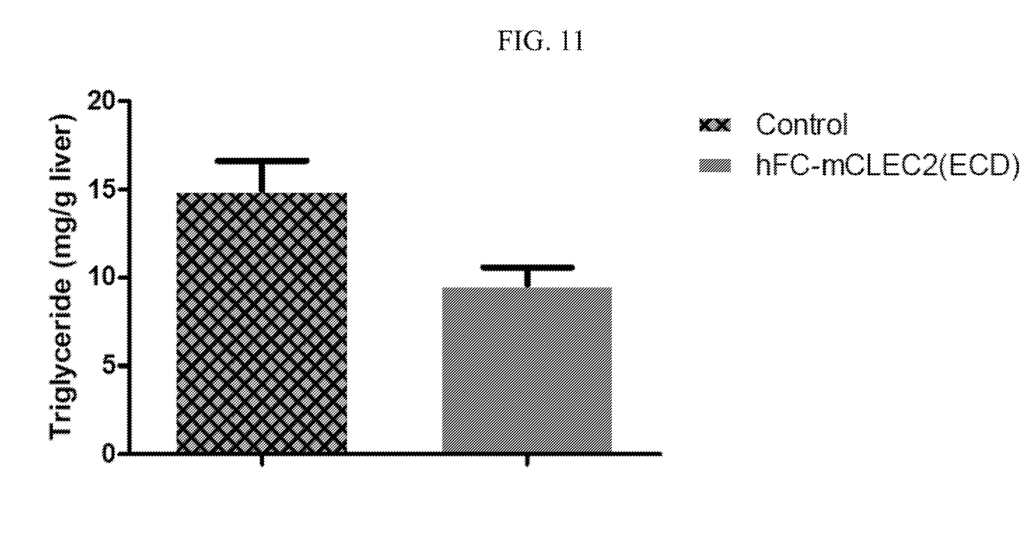

Liver triglyceride levels were measured 18 days post injection (FIG. 11) as follows: 2 ml of chloroform/methanol (2:1 v/v) was added to 40 to 50 mg of liver tissue and the tissues were homogenized in the Qiagen tissue lyzer for 30 seconds to 1 minute. Samples were then transferred to 12×75 mm glass test tubes and incubated at room temperature for 30-45 min. Samples were washed with 0.5 ml of 50 mM NaCl, vortexed, centrifuged at 1500 g or 2600 rpm for 10 minutes, and the organic phase was removed and placed into a new glass tube. The organic phase was washed with 0.5 ml of 0.36 M CaCl2/Methanol, vortexed, centrifuged at 1500 g or 2600 rpm for 10 minutes, and the organic phase was removed and place into a new glass tube. The organic phase was washed with 0.5 ml of 0.36 M CaCl2/Methanol, vortexed, centrifuged at 1500 g or 2600 rpm for 10 minutes, and the organic phase was removed and placed into a 2 ml volumetric flask. The volume was measured and sufficient chloroform was added to achieve a 2 ml volume. The triglyceride levels in the samples were measure using the Infinity triglyceride assay kit (Thermo Scientific, Catalog #TR22421).

The lowered blood glucose and insulin levels, triglyceride levels and improved oral glucose tolerance in the hFc-mCLEC2(ECD) HTV group compared to the control group demonstrated that HTV injected in vivo overexpression of hFc-mCLEC2(ECD) largely corrected metabolic abnormalities in DIO mice, including hyperglycemia, elevated triglyceride levels and hyperinsulinemia. This data confirmed our hypothesis that CLEC2 is involved in the regulation of body metabolism and that antagonism of CLEC2 can treat metabolic disorders, particularly diabetes.

### Example 4

### Recombinant Murine CLEC2 Extracellular Domain Fusion Protein Improves Glucose Tolerance and Lowers Insulin Level in DIO Mice

We tested the efficacy of recombinant hFc-mCLEC2(ECD) in a DIO model. Male 14-week-old B6D2F1 mice (From Jackson Lab, fed with Research Diets D12492 (60 kcal % fat) for 8 weeks) were divided into 3 groups (n=12) based on body weight and glucose levels. Starting from Day 0, the mice were IP-injected daily with control or 10 en mg/kg of hFC-mCLEC2(ECD) or 30 mg/kg of hFC-mCLEC2(ECD). The saline buffer control and hFC-mCLEC2(ECD) recombinant protein were prepared as described in Example 1. Protein was injected in 0.2 ml PBS. Injection usually occurred at about 4 PM (Dark Circle (lights out) starts at 6 PM). On the day of OGTT, proteins were given 2-3 hr before baseline glucose measurement and bleeding (10 AM).

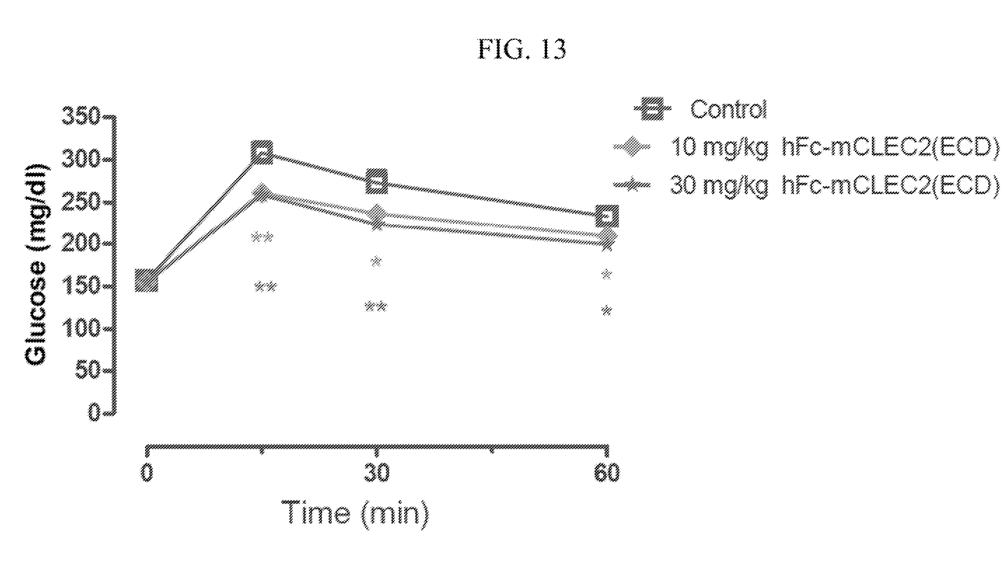

Glucose tolerance was measure 8 days after injection with of 10 mg/kg or 30 mg/kg of recombinant Fc-mCLEC2(ECD) or control. The GTT was performed as follows: animals were fasted for 4 hours. Following measurement of body weight and glucose levels (by glucometer), mice were injected with a bolus of glucose (10 ml/kg body weight of 20% glucose) into the stomach by a gavage needle. Glucose levels were measured with a glucometer by tail tip blood collection at 0, 15, 30 and 60 min after glucose dosing. FIG. 13 shows both the 10 mg/kg and 30 mg/kg recombinant hFc-mCLEC2(ECD) treated animals had lower glucose at every time point as compared to that of the control animals indicating improved glucose tolerance.

Plasma insulin levels were measured 15 days post injection of 10 mg/kg and 30 mg/kg of recombinant Fc-mCLEC2(ECD) or control, using the mouse Insulin ELISA kit from ALPCO Diagnostics (catalog number 80-INSMS-E01). Samples were diluted 1:10 in zero standard and 10 ul of diluted sample was used per well. The assay was performed as described in the manufacturer's protocol.

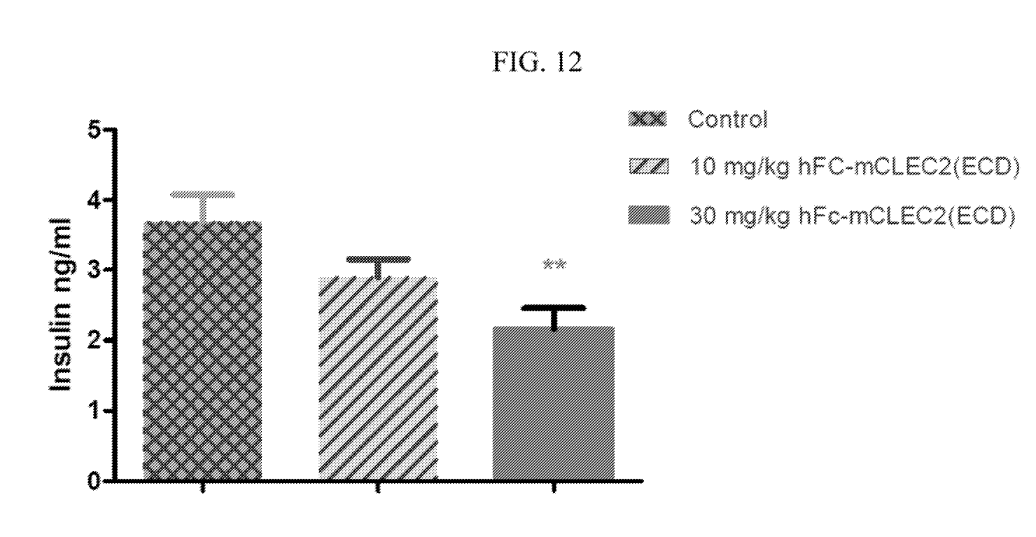

Mice treated with 10 mg/kg of recombinant Fc-mCLEC2(ECD) had a lower insulin level than that in the control animals and mice treated with 30 mg/kg of recombinant Fc-mCLEC2(ECD) had a lower insulin level than that of the control group and the 10 mg/kg treatment group (FIG. 12).

Collectively these results indicate that recombinant hFc-mCLEC2(ECD) is efficacious in DIO mice.

### Example 5

### AAV Mediated Overexpression of hFc-mCLEC2(ECD) Lowers Blood Glucose and Improves Glucose Tolerance in Ob/Ob Mice

Six week old male ob/ob mice (from Jackson Lab) were divided into 2 groups (n=12) based on body weight and baseline glucose. Group 1 was injected, through the tail vein, with control AAV, at 8¹² virus titer. The control was the empty vector that did not comprise hFc-mCLEC2. Group 2 was injected, through the tail vein, with AAV expressing hFc-mCLEC2, at 2¹² virus titer. The control and hFc-mCLEC2 rAAV construct are described in Example 2.

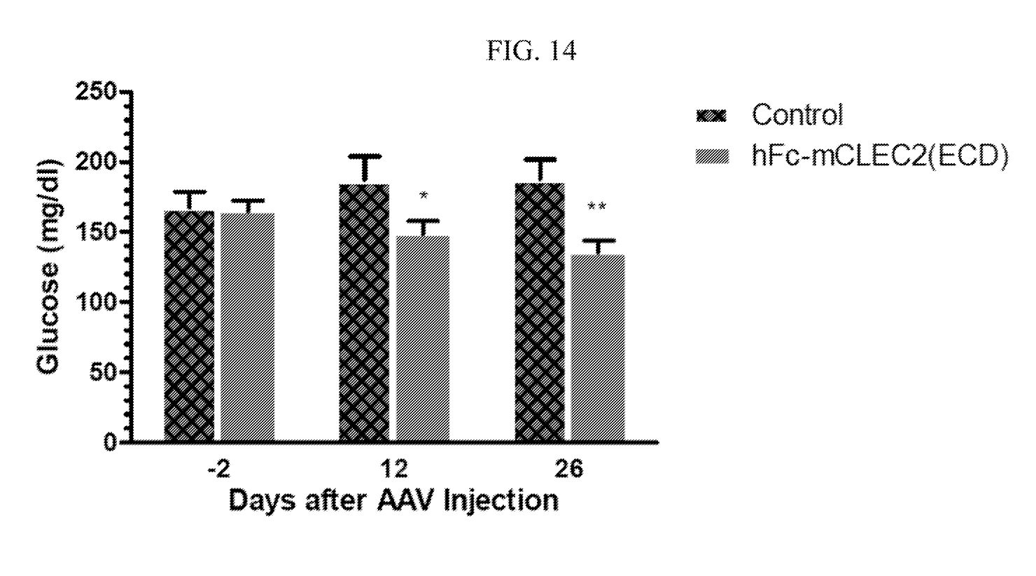

Blood glucose levels were measured two days prior to injection of the Fc-mCLEC2(ECD) or control and 12 days and 26 days post injection. Mice treated with Fc-mCLEC2(ECD) had a lower glucose level than that in the control at both day 12 and 26 post injection. (FIG. 14).

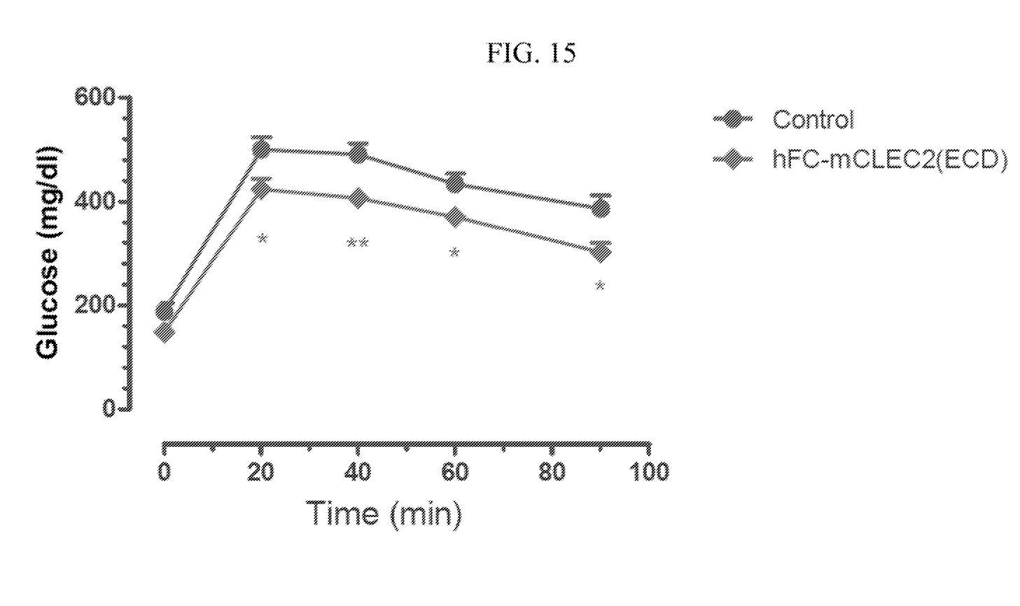

A glucose tolerance test (GTT) was performed 12 days after the AAV injection. The GTT was performed as follows: animals were fasted for 4 hours. Following measurement of body weight and glucose levels (by glucometer), mice were injected with a bolus of glucose (10 ml/kg body weight of 10% glucose) into the stomach by a gavage needle. Glucose levels were measured with a glucometer by tail tip blood collection at 0, 20, 40, 60 and 90 min after glucose dosing. FIG. 15 shows that hFc-mCLEC2(ECD) treated animals had lower glucose at every time point as compared to that of the control animals indicating improved glucose tolerance.

Collectively these results indicate that recombinant hFc-mCLEC2(ECD) is efficacious in Ob/Ob mice.

### Example 6

### Recombinant Hfc-Mclec2(Ecd Protein Improves Glucose Tolerance in Ob/Ob Mice

The efficacy of recombinant hFc-mCLEC2(ECD) protein was also tested in ob/ob mice. Six-week-old male ob/ob mice (Jackson lab) were divided into 2 groups (n=12) based on body weight and baseline glucose. Baseline glucose was measured by glucometer. Starting on day 1 through day 14, the mice were given an intraperitoneal injection with 10 ml/kg hFc-mCLEC2 (ECD) protein in PBS buffer or vehicle buffer.

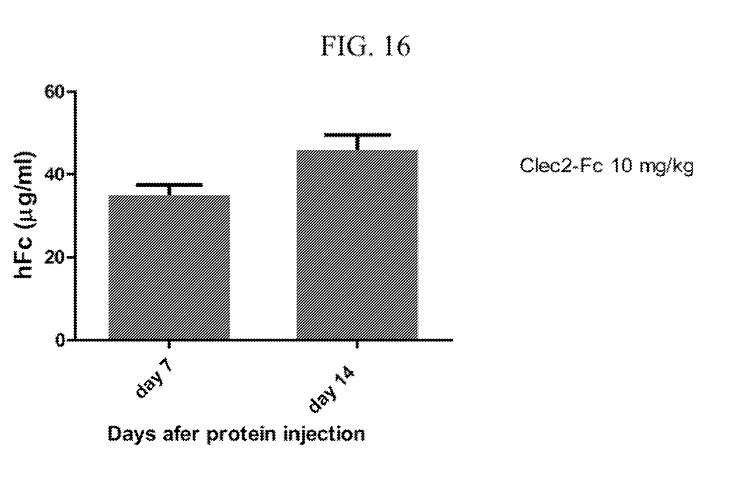

Serum protein levels of hFc-mCLEC2(ECD) were measured by hFc ELISA 7 and 14 days after injection as described in Example 2 (FIG. 16).

A glucose tolerance test (GTT) was performed 14 days after the AAV injection. The GTT was performed as follows: animals were fasted for 4 hours. Following measurement of body weight and glucose levels (by glucometer), mice were injected with a bolus of glucose (10 ml/kg body weight of 10% glucose) into the stomach by a gavage needle. Glucose levels were measured with a glucometer by tail tip blood collection at 0, 20, 30, 60 and 90 min after glucose dosing.

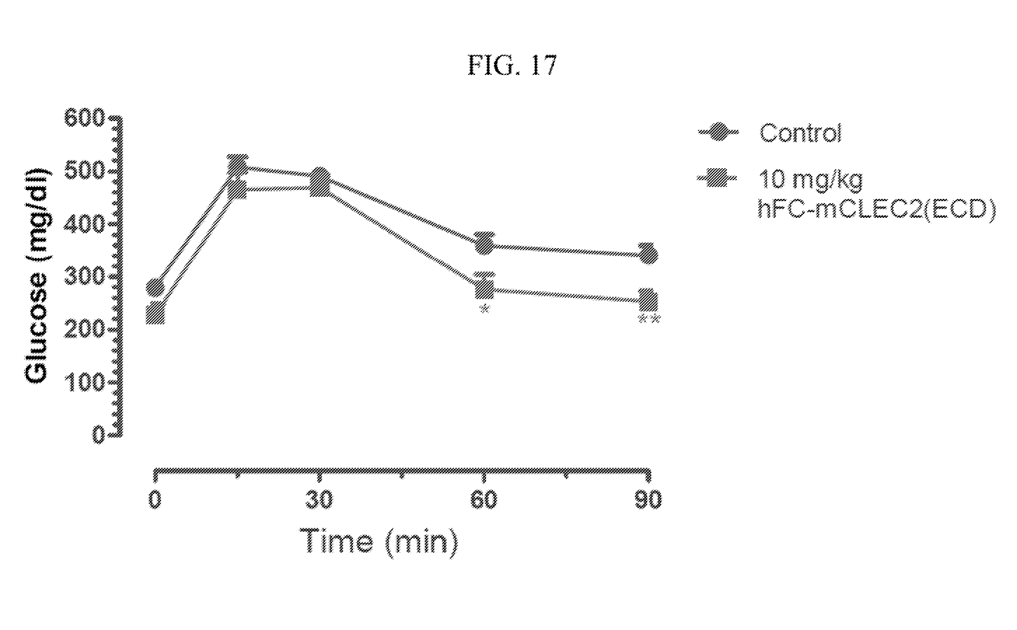

FIG. 17 shows that recombinant hFc-mCLEC2(ECD) treated animals had lower blood glucose levels at every time point as compared to that of the control animals indicating improved glucose tolerance.

### Example 7

### Preparation of Monoclonal Antibodies Specific to CLEC-2

A. Preparation of the Murine CLEC2 Immunogen

The FLAG epitope tagged version of murine or human CLEC2 polypeptide is used as an immunogen. The FLAG epitope is appended to the carboxy-terminus of murine or human CLEC2 using standard molecular biology techniques obvious to those skilled in the art. The epitope tagged version of CLEC2 is cloned into an expression vector for expression in HEK 293 and CHO cells. Other protein production and purification procedures known to those skilled in the art may also be used.

The CLEC2 immunogen may be the CLEC2 extracellular domain, see e.g. SEQ ID 3 or 9, or a fragment thereof with or without the FLAG epitope tag.

B. Immunization and Titering

HEK 293 cells expressing recombinant FLAG-tagged murine or human CLEC2 are used as antigen. Monoclonal antibodies against murine or human CLEC2 are developed by sequentially immunizing wild type C57BL/6 female mice. Animals are immunized via footpad and intraperitoneal routes for all injections. Anti-CLEC2 antibody titers in the serum from immunized mice are determined by FACS using CHO cells expressing recombinant FLAG-tagged CLEC2.

C. Recovery of Lymphocytes, B-Cell Isolations, Fusions and Generation of Hybridomas

B cells are harvested from lymph nodes and spleens of immunized mice. The fusion is performed by mixing washed B cells from above and nonsecretory myeloma P3X63Ag8.653 cells purchased from ATCC, catalogue CRL 1580 (Kearney et al, J. Immunol. 123, 1979, 1548-1550) at a ratio of 1:1. Electro-cell fusion (ECF) is performed using a fusion generator, model ECM2001, Harvard Apparatus, Inc., Holliston, Mass. The fusion chamber size used is 2.0 mL. After ECF, the cell suspensions were carefully removed from the fusion chamber under sterile conditions and transferred into a sterile tube containing the same volume of Hybridoma Culture Medium (DMEM based(Invitrogen)). The cells are incubated and then centrifuged. The cells are re-suspended in a small volume of Hybridoma Selection Medium (Hybridoma Culture Medium supplemented with 1× HAT (Sigma, catalogue H0262)), and the volume was adjusted appropriately with more Hybridoma Selection Medium. The cells are mixed gently and pipetted into 384-well plates and allowed to grow.

D. Hybridoma Screening

After sufficient culture, hybridoma supernatants are screened for CLEC2-specific monoclonal antibodies. In the Primary screen, hybridoma supernatants are incubated with CHO cells expressing recombinant FLAG-tagged murine or human CLEC2, and detection antibody FMAT Blue (Applied Biosystems) on FMAT plates for 3 hours at room temperature. After incubation, the plates are scanned with Applied Biosystems 8200 Cellular Detection System to detect positive hybridoma supernatants.

The old culture supernatants from the positive hybridoma cells growth wells based on primary screen are removed completely and the CLEC2 positive hybridoma cells are suspended with fresh hybridoma culture medium and transferred to 96-well plates. After 2 days of culture, a secondary confirmation screen is conducted where the positive hybridomas in the first screening are confirmed in FACS analyses. Two sets of CHO cells (one set expressing recombinant FLAG-tagged murine or human CLEC2 and the other not) are used in order to demonstrate specific binding. Selected hybridomas are expanded for antibody production and purification.

### Example 8

### Monoclonal Antibody in Murine DIO or Murine Ob/Ob Model

Six-week-old male B6D2F1 mice (from Jackson Lab) are housed in groups of four mice per cage and fed a high fat diet (up to 60% fat) for a period of up to 20 weeks. The baseline metabolic profile of body weight and glucose level are measured by collecting blood from the tail tip after four to six hours fasting.

Mice are selected and randomized into groups (n=12) based on body weight and baseline glucose. Mice are injected intraperitoneally (i.p.), 5 ml/kg, three times weekly. Group 1 is injected with vehicle only (A5Su: 10 mM Na-Acetate, 9% sucrose, pH5) Group 2 is injected with anti-Clec2 antibody. (20 mg/kg) and group 3 with anti-Clec2 antibody (20 mg/kg).

Serum protein levels of anti Clec2 antibodies are measured by an anti-Clec2 ELISA assay. Briefly, 96-well microtiter plate is coated with 1 ug/ml anti-huIgG Fc antibody overnight at 4 C. and washed in PBS twice. Recombinant mouse Clec2 protein are added at 1 ug/ml to the wells and incubated at room temperature for 1 hour. The plate are washed before diluted serum samples from treated mice are added to the wells. After one hour incubation, the wells are washed twice and HRP conjugated anti-mouse IgG antibody is added and incubated for 30 minutes. 100 ul of TMB solution is added to the well following two washes for color development at room temperature for about 15 minutes. 1N HCL is added to stop the color reaction. The plate is read in EnVision under OD450.

Oral glucose tolerance is measured after 7 and 14 days of treatment. Animals are fasted for 4 hours. Following measurement of body weight and glucose levels (by glucometer), mice are injected with a bolus of glucose (10 ml/kg body weight of 20% glucose) into the stomach by a gavage needle. Glucose levels are measured with a glucometer by tail tip blood collection at 0, 15, 30 and 60 min after glucose dosing . . . .

What is claimed is:

1. A method of treating a metabolic condition in a subject, comprising administering to the subject in need thereof a therapeutically effective amount of a Clec-2 inhibitor, wherein the Clec-2 inhibitor comprises the extracellular domain of the Clec-2 receptor or a fragment thereof.

2. The method of claim 1, wherein in the metabolic condition is diabetes, an elevated glucose level, an elevated insulin level, an elevated triglyceride level, insulin resistance or poor oral glucose tolerance.

3. The method of claim 2, wherein the diabetes is type II diabetes.

4. The method of claim 2 wherein the extracellular domain of the Clec-2 receptor or fragment thereof is the conjugated to an immuglobulin constant region or fragment thereof.

5. The method of claim 4, wherein the extracellular domain of the Clec-2 receptor or fragment thereof is a human extracellular domain or fragment thereof.

6. The method of claim 5 wherein the immunoglobulin constant region is a human immunoglobulin constant region or fragment thereof.

7. The method of claim 6, wherein the Clec-2 inhibitor comprises a polypeptide encoded by a polynucleotide which comprises a sequence having at least 90% sequence identity with that of SEQ ID 2, 4, 6 or 13.

8. The method of claim 7, wherein the Clec-2 inhibitor comprises a polypeptide encoded by a polynucleotide which comprises a sequence having at least 95% sequence identity with that of SEQ ID 2, 4, 6 or 13.

9. The method of claim 8, wherein the Clec-2 inhibitor comprises a polypeptide encoded by a polynucleotide which comprises a sequence having at least 98% sequence identity with that of SEQ ID 2, 4, 6 or 13.

10. The method of claim 9, wherein the Clec-2 inhibitor comprises a polypeptide is encoded by a polynucleotide having the sequence of SEQ ID 2, 4, 6 or 13.

11. The method of claim 6, wherein in the Clec-2 inhibitor comprises a polypeptide comprising an amino acid sequence at least 90% identical to the amino acid sequence of SEQ ID 1, 3, 5 or 12.

12. The method of claim 11 wherein in the Clec-2 inhibitor comprises a polypeptide comprising an amino acid sequence at least 95% identical to the amino acid sequence of SEQ ID 1, 3, 5 or 12.

13. The method of claim 12 wherein in the Clec-2 inhibitor comprises a polypeptide comprising an amino acid sequence at least 98% identical to the amino acid sequence of SEQ ID 1, 3, 5 or 12.

14. The method of 13 wherein in the Clec-2 inhibitor comprises a polypeptide comprising an amino acid sequence of SEQ ID 1, 3, 5 or 12.

15. The method of claim 6, wherein the subject is a mammal.

17. The method of claim 7, wherein the subject is a human.

18. The method of claim 18, wherein the Clec-2 inhibitor is administered in the form of a pharmaceutical composition comprising a therapeutically effective amount of the Clec-2 inhibitor in admixture with a pharmaceutically-acceptable carrier.

19. The method of claim 18, wherein following Clec-2 administration the subject comprises:
(a) a blood glucose level at a time point subsequent to administration of the Clec-2 inhibitor to the subject that is lower than at a time point prior to administration of the Clec-2 inhibitor,
(b) a plasma insulin level at a time point subsequent to administration of the Clec-2 inhibitor to the subject that is lower than at the time point prior to the administration.
c) an insulin resistance that is improved at a time point subsequent to administration of the Clec-2 inhibitor to the subject than at a time point prior to the administration,
(d) a blood triglyceride level at a time point subsequent to administration of the Clec-2 inhibitor to the subject that is lower than at a time point prior to the administration, or
(e) an oral glucose tolerance that is improved at a time point subsequent to administration of the Clec-2 inhibitor than at a time point prior to the administration.
